(19) United States
(12) Patent Application Publication
(10) Pub. No.: US 2023/0303806 A1
(43) Pub. Date: Sep. 28, 2023

(54) COMPOSITE FILM, SENSOR ELEMENT COMPRISING SAID COMPOSITE FILM, BODY FAT PERCENTAGE MEASURING DEVICE, AND ELECTROCHEMICAL CELL DEVICE, AND WEARABLE MEASURING DEVICE COMPRISING SAID SENSOR ELEMENT

(71) Applicant: UNIVERSITY PUBLIC CORPORATION OSAKA, Osaka-shi, Osaka (JP)

(72) Inventor: Hiroshi SHIIGI, Sakai-shi, Osaka (JP)

(73) Assignee: UNIVERSITY PUBLIC CORPORATION OSAKA, Osaka-shi, Osaka (JP)

(21) Appl. No.: 18/041,505

(22) PCT Filed: Jul. 30, 2021
(86) PCT No.: PCT/JP2021/029241

§ 371 Date: Feb. 13, 2023

(30) Foreign Application Priority Data
Aug. 14, 2020 (JP) 2020-136975

(51) Int. Cl. C08L 1/02; A61B 5/0537; A61B 5/1486

(52) U.S. Cl. CPC C08L 1/02; A61B 5/0537; A61B 5/1486; C08L 2203/16; C08L 2205/16

(57) ABSTRACT

The present invention provides a composite film that has electrical conductivity, mechanical strength and flexibility which resist being affected by moisture and are stable, and that can prevent position aberration and peeling in the case where the composite film is used in close contact with a body to be contacted; a sensor element, a body fat percentage measuring device, and an electrochemical cell device which are provided with the composite film; and a wearable measuring device including the sensor element. The composite film includes electroconductive nanoparticles and nanofibers, wherein the nanofibers have a plurality of gaps therebetween that are communicated with an outside; the electroconductive nanoparticles adhere to the surface of the nanofibers and exist in the plurality of gaps; the nanofibers are hydrophilic and biocompatible; and the composite film is electroconductive and is used in close contact with a body to be contacted that is hydrophilic-treated or that contains moisture.

### TECHNICAL FIELD

The present invention relates to a composite film; a sensor element comprising the composite film, a body fat percentage measuring device and an electrochemical cell device; and a wearable measuring device comprising the sensor element.

### BACKGROUND ART

In recent years, with the development of biotechnology, attention has been focused on the development of a technology relating to the evaluation and utilization of biological functions. The biological functions have each established specificity. Then, biosensors are being developed and utilized which use a non-biological material such as an electronic device. The biosensors capable of performing analysis quickly, conveniently, and at low cost are very useful; and application research using nanotechnology techniques is being actively carried out.

For example, in patent literature 1, a detection device is disclosed in which a main body of a cell is filled with a solution of a mixture containing an enzyme body. The detection device in patent literature 1 detects a target substance by using a molecular recognition function which an enzyme has. As an electrode of such a biosensor, an electroconductive pattern (electrode or circuit) is used which is a metal thin film. In general, the electroconductive pattern is formed with the use of a method such as screen printing, electroless plating, sputtering or vapor deposition, as the metal thin film on a flexible material.

In addition, in patent literature 2, a composite film is disclosed which is derived from a material containing cellulose nanofibers and metal nanoparticles. However, in patent literature 2, a specific mode of use is not disclosed, at the time when a composite film is used as an electrode for evaluation of the biological function.

### CITATION LIST

### Patent Literature

### Patent Literature 1: Japanese Patent Laid-Open No. 2016-208883

### Patent Literature 2: Japanese Patent Laid-Open No. 2018-154921

### SUMMARY OF INVENTION

### Technical Problem

In order to develop a biosensor which exhibits excellent characteristics and can easily detect a very small amount of a target substance, it is extremely important to develop a material of which the specificity and sensitivity are enhanced, and besides which can be stably and easily produced. For example, an electrode which is obtained by printing an electroconductive ink on a plastic sheet or the like is useful for obtaining biological information. However, when the electrode is used on a living body, a measurement error or the like occurs which is based on position aberration of the electrode due to movement and a change in sweating amount. Because of this, it becomes necessary to miniaturize the electrode, and develop a system which corrects a deviation of a measured value due to sweating, or the like. For example, various metabolites and electrolytes contained in sweat have a correlation with blood. Because of this, the development of sensors targeting these metabolites has attracted much attention in a field of wearable devices. In addition, when the electrode is miniaturized, the information which is acquired by one electrode becomes local. Because of this, when information is acquired from a wide range, multi-point measurement becomes necessary; and it becomes necessary to further devise a design of the wearable device.

In addition, there are cases where biological substances which living organisms produce are unstable or can only be produced in limited environments. In other words, it is preferable to measure the biological substance in an environment as close to the living body as possible. Accordingly, the electrode as the wearable device is desirably excellent in flexibility enough to follow the movement of the living body, in order to precisely acquire information from the living body, and is also required to have such a mechanical strength as not to be broken by the movement of the living body. In addition, the electrode as the wearable device is brought into direct contact with the living body, and accordingly is desirable to maintain air permeability, and safety to the human body and the like.

Accordingly, an object of the present invention is to provide a composite film that has electrical conductivity, mechanical strength and flexibility which are hardly affected by moisture and stable, and that can prevent position aberration and peeling in the case where the composite film is used in close contact with the body to be contacted; a sensor element comprising the composite film, a body fat percentage measuring device, and an electrochemical cell device; and a wearable measuring device comprising the sensor element.

### Solution to Problem

The present inventors have made an extensive investigation in order to achieve the above object, and as a result, have found that a composite film including an electroconductive nanoparticle and a hydrophilic nanofiber has stable electrical conductivity which is not affected by the amount of moisture. The present invention has been completed on the basis of these findings.

Specifically, the present invention provides a composite film including electroconductive nanoparticles and nanofibers, wherein the nanofibers have a plurality of gaps therebetween that are communicated with an outside; the electroconductive nanoparticles adhere to the surface of the nanofibers and exist in the plurality of gaps; the nanofibers are hydrophilic and biocompatible; and the composite film is electroconductive and is used in close contact with a body to be contacted that is hydrophilic-treated or that contains moisture.

It is preferable that the amount of the electroconductive nanoparticles is 2.0 to 20 vol. % with respect to the total amount (100 vol. %) of the electroconductive nanoparticles and the nanofibers.

It is preferable that the nanofiber contains cellulose.

It is preferable that the electroconductive nanoparticle includes a metal, a metal oxide, or carbon.

It is preferable that the tensile strength of the composite film is 0.5 to 100 MPa.

It is preferable that the body to be contacted is skin or a tissue in a living body.

It is preferable that the body to be contacted includes metal, glass, plastic, ceramic, or carbon.

It is preferable that the composite film has flexibility due to which the composite film is deformed or expands or contracts in accordance with the movement of the human body when having been attached to the human body, and shows a change in a resistance value caused by the movement of the human body of 2.0Ω or smaller.

In the composite film, it is preferable that a change in the resistance value caused by an increase or decrease of a liquid existing in the plurality of gaps is 0.5Ω or smaller.

The present invention provides a sensor element including the composite film and a molecular recognition body arranged in the plurality of gaps.

It is preferable that the molecular recognition body includes an enzyme, an antibody, DNA or RNA containing an aptamer, an artificial antibody formed from a molecularly imprinted polymer, or an ion-selective molecule.

It is preferable that the enzyme includes an oxidase, a reductase, or a dehydrogenase.

It is preferable that the oxidase includes glucose oxidase or lactate oxidase.

It is preferable that the dehydrogenase includes glucose dehydrogenase or lactic acid dehydrogenase.

The present invention provides a wearable measuring device comprising the sensor element.

The present invention provides a body fat percentage measuring device comprising the composite film.

The present invention provides an electrochemical cell device comprising the composite film.

### Advantageous Effects of Invention

The composite film according to the present invention has stable electrical conductivity, mechanical strength and flexibility which resist being affected by moisture, and when being used in close contact with the body to be contacted, can prevent position aberration and peeling. In addition, the electroconductive nanoparticles in the composite film can be easily recovered and repeatedly used.

### BRIEF DESCRIPTION OF THE DRAWINGS

FIG. 1 shows a schematic view of a body fat percentage measuring device according to one embodiment of the present invention.

FIG. 2(A) shows a schematic cross-sectional view of a sensor element according to one embodiment of the present invention, and FIG. 2(B) shows a schematic cross-sectional view of a conventional sensor element in which enzymes are immobilized on a flat plate.

FIG. 3 shows an electron micrograph of a composite film according to Example 2.

FIG. 4(A) shows a graph showing a temporal change in moisture content at the time when the composite film according to Example 3 has been immersed in water, and FIG. 4(B) shows a graph showing a relationship between elapsed time and a resistance value at the time when the composite film has been immersed in water.

FIG. 5(A) shows a graph showing a relationship between a concentration of gold contained in an AuNP/CNF film and specific resistivity, and FIG. 5(B) shows a graph showing a relationship between the concentration of gold contained in the AuNP/CNF film and tensile strength.

FIG. 6(A) shows a voltammogram obtained by CV measurement of a composite film according to Example 6 in a solution of K₃[Fe(CN)₆], and FIG. 6(B) shows a plot of peak current values with respect to a volume occupancy of gold in the AuNP/CNF film.

FIG. 7 shows a voltammogram obtained by the CV measurement of the composite film according to Example 6 in a solution of 0.1 M KCl.

FIG. 8 shows a voltammogram obtained by CV measurements before and after cleaning of a composite film according to Example 7.

FIG. 9(A) shows a result of CV measurement with the use of a composite film according to Example 9, and FIG. 9(B) shows a graph obtained by plotting peak current values with respect to a square root of a sweep rate from the result of FIG. 9(A). FIG. 9(C) shows the result of CV measurement with the use of a gold disk electrode, and FIG. 9(D) shows a graph obtained by plotting peak current values with respect to a square root of a sweep rate from the result of FIG. 9(C).

FIG. 10(A) shows an absorption spectrum of a glucose solution of Reference Example 1 by a colorimetric quantitative method, and FIG. 10(B) shows a graph obtained by plotting absorbance at 505 nm with respect to the glucose concentration in a measuring cell, on the basis of the result of FIG. 10(A).

FIG. 11(A) shows a graph of a current response at the time when a solution of 25 mM glucose according to Example 11 has been added every minute, and FIG. 11(B) shows a graph obtained by plotting the current values with respect to the concentration, on the basis of the result of FIG. 11(A).

FIG. 12(A) shows a graph of a current response at the time when a low concentration of glucose has been measured in Example 11, and FIG. 12(B) shows a graph obtained by plotting the current values with respect to the concentration on the basis of the result of FIG. 12(A).

FIG. 13(A) shows a graph of a current response at the time when each solution according to Example 12 has been added, and FIG. 13(B) shows a graph obtained by plotting the current values with respect to glucose concentration, on the basis of the result of FIG. 13(A).

FIG. 14 shows a schematic view of a two-electrode cell according to Example 13.

FIG. 15(A) shows a graph of a current response at the time when a glucose solution according to Example 13 has been added, and FIG. 15(B) shows a graph obtained by plotting peak current values with respect to the concentration, on the basis of the result of FIG. 15(A).

FIG. 16(A) shows a graph showing the timing of sweat collection according to Example 14, FIG. 16(B) shows a result of amperometry which has used sweat before and after a meal, and FIG. 16(C) shows a graph obtained by plotting the current values with respect to elapsed time after a meal.

FIG. 17(A) shows a graph of a current response at the time when a lactic acid solution according to Example 17 has been added, and FIG. 17(B) shows a graph obtained by plotting current values with respect to the concentration, on the basis of the result of FIG. 17(A).

FIG. 18(A) shows a graph of a current response at the time when each solution according to Example 18 has been added, and FIG. 18(B) shows a graph obtained by plotting the current values with respect to a lactic acid concentration, on the basis of the result of FIG. 18(A).

FIG. 19(A) shows a graph of a current response at the time when a lactic acid solution according to Example 19 has been added, and FIG. 19(B) shows a graph obtained by plotting the current values with respect to the concentration, on the basis of the result of FIG. 19(A).

FIG. 20(A) shows a photograph showing a state in which an AuNP/CNF film according to Example 20 has been attached to the palm, and FIG. 20(B) shows a graph showing a change in the resistance value of the AuNP/CNF film at the time when the palm is opened and closed every 1 second.

FIG. 21 shows a schematic view showing one embodiment of a two-electrode cell which uses the composite film of the present invention.

FIG. 22 shows a schematic view showing another embodiment of a two-electrode cell which uses the composite film of the present invention.

FIG. 23 shows a schematic view showing one embodiment of a three-electrode cell which uses the composite film of the present invention.

FIG. 24 shows a schematic view showing another embodiment of a three-electrode cell which uses the composite film of the present invention.

FIG. 25 shows a voltammogram obtained by CV measurement in Example 21.

FIG. 26 shows a voltammogram obtained by CV measurement in Example 22.

### DESCRIPTION OF EMBODIMENTS

### [Composite Film]

A composite film according to one embodiment of the present invention (hereinafter, also simply referred to as “composite film”) includes an electroconductive nanoparticle and a nanofiber, and has electrical conductivity. In the composite film, a plurality of nanofibers is layered randomly, for example, and forms layers. For example, the composite film is a nonwoven fabric made of nanofibers. For information, the composite film is not limited to nonwoven fabric, and maybe a woven fabric, a knitted fabric, or the like, any of which is formed from a yarn containing the nanofiber.

The nanofibers form a plurality of gaps between the nanofibers. As a result, the composite film has a plurality of gaps that communicates with the outside, and accordingly has a structure excellent in air permeability. In addition, the composite film has such a structure that a liquid such as water can enter and exit from the gap of the composite film, and thereby, the composite film can be cleaned up to the inside, and can be repeatedly used.

The composite film can be used in a state of being brought into close contact with a body to be contacted that is hydrophilic-treated or that contains moisture. Examples of the body to be contacted in a state before the hydrophilic treatment in the hydrophilic-treated body to be contacted include metal, glass, plastic, ceramic, and carbon. When the body to be contacted is glass, examples of the hydrophilic treatment include plasma treatment. When the body to be contacted is a metal, examples of the hydrophilic treatment include treatment which uses a thiol compound. In the treatment which uses the thiol compound, a solution (an aqueous solution or the like) is applied to a metal surface, which contains a compound having a moiety that can form a bond with the cellulose nanofiber or the like in the composite film, such as a carboxyl group having a thiol group, an amino group or a hydroxy group, and a moiety that can form a bond with metal; and the resultant metal surface is left to stand. After that, it is acceptable to clean the metal surface with ultrapure water, and remove the excess solution. In the case where the body to be contacted is carbon, examples of the hydrophilic treatment include electrolytic polishing of the surface of the body to be contacted in an acidic or alkaline aqueous solution, or hydrophilic treatment with an additive such as a surface-active agent. The composite film uses a hydrophilic nanofiber, and accordingly, when having been used in close contact with a hydrophilic-treated body to be contacted, the composite film can prevent position aberration and peeling. A product obtained by a combination of the composite film with another material can be used as a layered body having properties of another material as well. For information, it is also possible to use not only one surface of the composite film but also both surfaces of the composite film, by bringing the surfaces into close contact with the hydrophilic-treated bodies to be contacted, respectively.

On the other hand, examples of the body to be contacted that contains moisture include a living body, wood, and a plant. The body to be contacted that contains moisture can attach the composite film to its surface by moisture which oozes out from the body to be contacted itself. In the case where the body to be contacted is a living body, the composite film can absorb components oozing from the surface of the skin in the gap, by directly being brought into close contact with the skin or the like. For example, when sweat enters the gap of the composite film, the sweat expels air existing in the gap of the composite film, and the composite film can adhere to the surface of the skin. In addition, the composite film can also be used in the body. For example, when the composite film is attached to the palate in the oral cavity, saliva enters the gap of the composite film and expels air existing in the gap of the composite film, and thereby the composite film can be attached to the palate. In addition, the composite film has flexibility, and accordingly can be brought into close contact with an object having a stereoscopic effect as well, such as a human body. Thereby, the composite film can be brought into close contact even with a portion on which there are, for example, irregularities such as wrinkles of the palm or the like. For information, it is also possible to use not only one surface of the composite film but also both surfaces of the composite film, by bringing the surfaces into close contact with a moisture-containing body to be contacted. In addition, it is also possible to use the composite film by bringing its one surface into close contact with a hydrophilic-treated body to be contacted, and the other surface into close contact with a hydrophilic-treated body to be contacted, respectively.

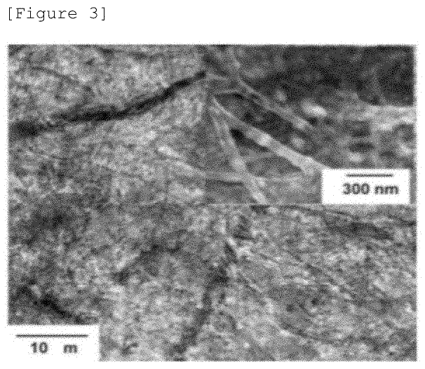

The electroconductive nanoparticle adheres to the surface of the nanofiber. The electroconductive nanoparticle is bonded to the nanofiber by, for example, a hydrogen bond. When the above nanofiber is cellulose, the hydrogen bond is formed between the electroconductive nanoparticle and the cellulose, for example, via a binder which will be described later. Among the above binders, a carboxylic acid (salt) is preferable for the purpose of forming the above hydrogen bond by a hydroxy group of the cellulose, and citric acid (salt) is particularly preferable from the viewpoint of being more excellent in biocompatibility. The electroconductive nanoparticle exists in a state of entering into the gap between the nanofibers. When metal nanoparticles are used as electroconductive nanoparticles, the metal nanoparticles preferably exist side by side so as to be connected along the axial direction of the nanofiber, for example, as will be shown in FIG. 3 of Example 2 which will be described later. When the metal nanoparticles exist along a bundle of nanofibers while being connected, the metallic electrical conductivity as the composite film is more sufficiently ensured. In other words, when the metal nanoparticles exist in the composite film in a state of being continuously arranged, an electron can smoothly move between adjacent metal nanoparticles. Accordingly, the electrical conductivity of the composite film is ensured by a much smaller amount of metal nanoparticles than the amount in a structure in which metal nanoparticles uniformly exist. For information, the same effect can be expected when the above structure is formed (in which electroconductive nanoparticles exist side by side so as to be connected along the axial direction of the nanofiber), also in the case where electroconductive nanoparticles such as particles of metal oxide and carbon has been employed in place of the metal nanoparticle.

The film thickness of the composite film can be appropriately set according to the application and a required function, but is preferably, for example, 0.05 to 20 μm. When the film thickness of the composite film is 0.05 μm or larger, the composite film obtains sufficient mechanical strength and has self-standing properties. When the film thickness of the composite film is 20 μm or smaller, the composite film can obtain sufficient flexibility. A method for measuring the film thickness of the composite film will be described in detail in Example 4.

It is preferable for the amount of the electroconductive nanoparticle in the composite film to be 2.0 to 20 vol. %, is more preferable to be 6.0 to 18 vol. %, and is further preferable to be 10 to 17 vol. %, with respect to the total amount (100 vol. %) of the electroconductive nanoparticle and the nanofiber. When the amount of the electroconductive nanoparticles in the composite film is 2.0 vol. % or more with respect to the total amount of the electroconductive nanoparticle and the nanofiber, sufficient electrical conductivity can be obtained. When the amount of the electroconductive nanoparticles in the composite film is 20 vol. % or less with respect to the total amount of the electroconductive nanoparticle and the nanofiber, sufficient flexibility of the composite film can be obtained.

It is preferable for the specific resistivity of the composite film to be 1×10⁻³ Ωcm or smaller, is more preferable to be 1×10⁻⁴ Sim or smaller, and is further preferable to be 1×10⁻⁵ Ωcm or smaller. The specific resistivity of the composite film depends on the metal content of the composite film, specifically, the content of the electroconductive nanoparticle, and the state of the composite. The specific resistivity of gold which is used as the electroconductive material is, for example, 2.44×10⁻⁶ Ωcm. A composite film having a specific resistivity of 1×10⁻³ Ωcm or smaller is suitable as the electroconductive material. A method for measuring the specific resistivity of the composite film will be described in detail in Example 8.

It is preferable that the composite film has the flexibility due to which the composite film is deformed or expands or contracts in accordance with the movement of the human body, when having been attached to the human body. As a result, when the composite film is used in close contact with the body to be contacted, position aberration and peeling are less likely to occur. In addition, it is preferable for a change in a resistance value of the composite film caused by movement of the human body to be 2.0Ω or smaller, is more preferable to be 1.5Ω or smaller, and is further preferable to be 1.2Ω or smaller. When the change in the resistance value is 2.0Ω or smaller, in the case where the composite film is used, for example, as an electrode, an obtained current value is less likely to be affected by the movement of the human body, and accordingly, an accurate value can be obtained. For information, examples of the movement of the human body includes, opening and closing of the palm when the composite film is attached to the palm, and movement of the joint when the composite film is attached to the joint such as the elbow.

It is preferable for a change in the resistance value of the composite film caused by the increase or decrease of a liquid existing in the plurality of gaps to be 0.5Ω or smaller, is more preferable to be 0.4Ω or smaller, and is further preferable to be 0.3Ω or smaller. When the change in the resistance value is 0.5Ω or smaller, in the case where the composite film is used, for example, as an electrode, an obtained current value is less likely to be affected by the amount of a liquid existing in the gap of the composite film, or a use environment such as humidity, and accordingly, an accurate value can be obtained.

It is preferable for a tensile strength of the composite film to be 0.5 to 100 MPa, is more preferable to be 5 to 80 MPa, and is further preferable to be 10 to 60 MPa. The tensile strength of the composite film depends on a content of the electroconductive nanoparticles in the composite film, and a state of the composite. When the tensile strength of the composite film is 0.5 MPa or higher, the film is less likely to be damaged, and has sufficient durability even when the film is attached, for example, to the human body or the like and is used. When the tensile strength of the composite film is 100 MPa or lower, the flexibility is high, in the case where the composite film is attached, for example, to the human body or the like and is used, the composite film can be deformed or expand or contract in accordance with the movement of the human body, and accordingly can prevent the position aberration and the peeling. In addition, a method for measuring the tensile strength of the composite film will be described in detail in Example 4.

For information, the metallic glossiness (reflectance) of the composite film may not be particularly required. The reflectivity of the composite film may be, for example, lower than 50% of the total reflectivity of the pure metal foil. Because of this, the composite film does not need to be subjected to processes of hot press or plating for improving the metallic glossiness, and the composite film can be easily produced.

(Nanofiber)

The nanofiber is hydrophilic and has biocompatibility. In the present specification, “biocompatibility” refers to the property of being harmless and keeping safety when a substance is brought into contact with a living body such as the human body. Examples of nanofiber include substances that contain cellulose, chitosan, chitin and other polysaccharides as raw materials. Polysaccharides have a large number of hydroxyl groups in the molecule, and accordingly, have an affinity with water. Furthermore, these nanofibers have amphiphile properties of having also hydrophobicity, and accordingly, exhibit sufficient mechanical strength even at the time when containing moisture. A cellulose nanofiber (hereinafter, also referred to as CNF) is preferable as the nanofiber, particularly from the viewpoint of availability and safety to a living body. In addition, a composite film obtained from the cellulose nanofiber is provided with mechanical strength and flexibility. Furthermore, when the electroconductive nanoparticle is an inorganic component such as metal nanoparticles, the cellulose nanofiber can be separated from the electroconductive nanoparticle by combustion after use. Because of this, the electroconductive nanoparticle contained in the composite film can be easily recovered and reused after use, and can be used repeatedly in some cases.

The cellulose nanofiber is formed of a polysaccharide, for example, in which glucose is bonded to β-1,4-glycoside. In addition, the cellulose nanofiber is a fiber having a fiber diameter, for example, of 1 to 100 nm. The cellulose nanofiber which is used in the present embodiment is not particularly limited as long as the cellulose nanofiber can be complexed with the electroconductive nanoparticle; and include known cellulose nanofibers, for example, cellulose nanofibers which are obtained by bacteria synthesis, and extracts from natural products such as plants and processed products thereof. In particular, the former can be obtained as a nanofiber film having a desired film thickness, when the synthesis conditions are set. On the other hand, in the case of the latter, as will be described in a production method, a solution containing the cellulose nanofiber can be formed into a nanofiber film having a desired film thickness by a method such as suction filtration.

It is preferable that the cellulose nanofiber is a film of a bacterial cellulose nanofiber or a cellulose nanofiber derived from a plant, because of the ease of synthesis and availability. As the cellulose nanofiber is derived from a plant, commercially available solutions containing cellulose nanofibers can be used as will be described in Example. In the present invention, the cellulose nanofiber contributes to the mechanical characteristics of the composite film. In other words, the mechanical characteristics of the cellulose nanofiber are utilized, and accordingly, it becomes possible to control the mechanical characteristics of the composite film with high accuracy by an aspect ratio, through a microfabrication process for uniformizing the fiber lengths and the fiber diameters (together with aspect ratio) is not particularly required. For example, the mechanical strength and the flexibility of the composite film can be adjusted according to specifications. In the present embodiment, a cellulose nanofiber may also be suitably used which has such a low degree of dissociation as to cause white turbidity when having formed a dispersion solution. For information, the cellulose nanofiber may contain a nanofiber other than the cellulose, as long as the function of the present invention is not impaired.

(Electroconductive Nanoparticle)

In the present specification, the electroconductive nanoparticle shall refer to a particle that has a size on the order of nanometers and has electrical conductivity. The order of nanometers includes a range of 1 to hundreds of nanometers, and typically the particle size is in a range of 1 to 100 nm.

An average particle diameter (median diameter, D50) of the electroconductive nanoparticles is not particularly limited, but is preferably 15 to 100 nm, and is more preferably 15 to 50 nm. When the average particle diameter of the electroconductive nanoparticles is 15 nm or larger, the compatibility with the cellulose nanofiber decreases, and the mechanical strength of the composite film is enhanced. In addition, it is possible to suppress the amount of the electroconductive nanoparticles to be used for obtaining sufficient electrical conductivity, to a predetermined amount or less. When the average particle diameter of the electroconductive nanoparticles is 100 nm or smaller, the compatibility with the cellulose nanofiber is enhanced, the coagulation of the electroconductive nanoparticles is suppressed, and a uniform composite film is formed. In addition, it is possible to suppress the amount of the electroconductive nanoparticles to be used for obtaining sufficient electrical conductivity, to a predetermined amount or less. The average particle diameter of the electroconductive nanoparticles is a value obtained by a number average, and can be determined from an average value of particle diameters of arbitrary 100 electroconductive nanoparticles, which have been measured, for example, from an image photographed with the use of a transmission electron microscope.

The electroconductive nanoparticle to be used in the present embodiment is not particularly limited, as long as the nanoparticle can be combined with the nanofiber, and may be appropriately selected depending on the application of the composite film and the required function. Examples of components constituting the electroconductive nanoparticle include a metal, a metal oxide, and carbon. For information, the electroconductive nanoparticle may be composed of only one type of component or may contain a plurality of types of components. The electroconductive nanoparticle is preferably a particle which contains a metal (in other words, metal nanoparticle) among the components, as a constituent component. The metal nanoparticle may be, for example, a nanoparticle that is formed from a single element such as gold, silver, palladium, platinum, nickel, copper, iron, lead, lithium, cobalt, manganese, aluminum, zinc, bismuth, silicon, tin, cadmium, indium, titanium and tungsten, a nanoparticle that is formed from a plurality of elements of these metals, a nanoparticle that includes oxides or salts of these metals, or a nanoparticle that includes an electroconductive substance other than metals, such as a carbon particle. When the composite film is attached to the human body and used, the electroconductive nanoparticle is preferably a metal nanoparticle selected from gold, silver, palladium, and platinum, for example. These metal nanoparticles give relatively little influence on the human body, and can impart electrical conductivity to the composite film. When the composite film is used for a device such as an electrochemical cell device including a battery or an electrolytic cell, the electroconductive nanoparticle may employ, for example, a nanoparticle that is formed from a single element such as nickel, copper, iron, lead, lithium, cobalt, manganese, aluminum, zinc, bismuth, silicon, tin, cadmium, indium, titanium and tungsten, a nanoparticle that is formed from a plurality of elements of these metals, a nanoparticle that includes oxides or salts of these metals, or a nanoparticle that includes an electroconductive substance other than metals such as a carbon particle, if the nanoparticles do not affect the performance of the device.

In particular, a gold nanoparticle has little influence of allergy or the like on the human body, and accordingly, the composite film can be safely used in close contact with the skin. Accordingly, the composite film can be safely used also for the skin or the tissue inside the living body. For information, the gold nanoparticle can be produced by a known method, for example, a method described in International Publication No. WO2010/095574.

(Applications)

The composite film has electrical conductivity, high strength, excellent heat resistance and flexibility, self-standing properties, and is easy of in-mold forming and pattern forming; and can be expected to be used not only for optical and electronic materials but also for new applications such as electrode materials, sensor elements, wearable materials and electromagnetic wave protective materials. For example, the composite film can be used also in a body fat percentage measuring device and the like as will be described in detail below. In addition, the composite film is a material safe for the living body, and accordingly, can be used not only in a state of being attached to the surface of the body, but also in the body such as an oral cavity or an organ. For example, the composite film can be used as a material that is used in a surgical operation. In addition, by being combined with another material, the composite film can be used also for an electrochemical cell device and the like which include a battery or an electrolytic cell.

When the electrochemical cell device is used as a battery or an electrolytic cell, the composite film functions as a current collector or an electrode. The composite film can be attached directly to the inside of the hydrophilic-treated battery or electrolytic cell. Thereby, the battery or the electrolytic cell can be reduced in weight and thickness. In addition, the composite film is directly attached to the battery or the electrolytic cell, accordingly, the arrangement of the composite film can be facilitated, and the production process can be simplified. In addition, the composite film has a larger surface area than that of the metal thin film because of having the gap. In general, in the electrochemical cell device, as the surface area of an electrode increases, a reaction region becomes wider in which electrons can move. Because of this, when the electrochemical cell device is used as a battery or an electrolytic cell, the composite film can ensure a large surface area, and can enhance the capacity and reaction efficiency of the battery.

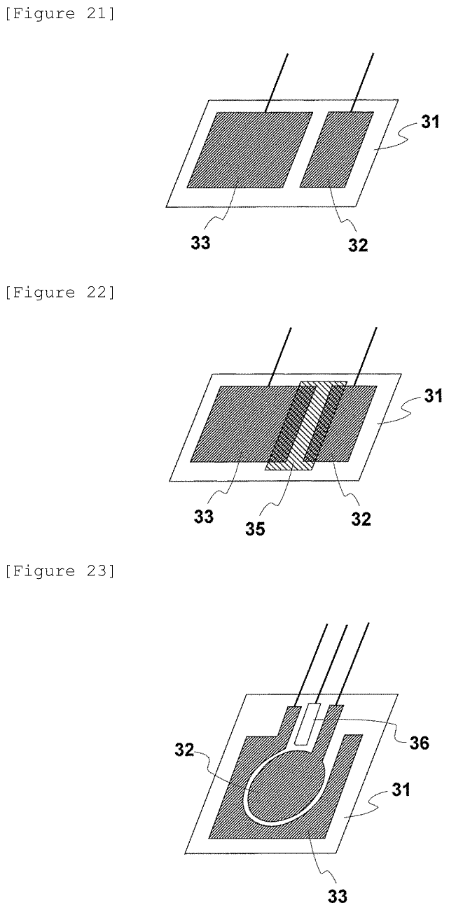

Examples of the battery or electrolytic cell using the above composite film include a two-electrode cell and a three-electrode cell. One embodiment of each of the above two-electrode cell and the above three-electrode cell is shown in FIG. 21 to FIG. 24. The two-electrode cells shown in FIG. 21 and FIG. 22 include a base material 31, and a working electrode 32 and a counter electrode 33 which are installed on the base material 31. In the two-electrode cell shown in FIG. 21 and FIG. 22, the working electrode 32 is an electrode formed of the above composite film, and the electrode is used by impregnating, for example, the base material 31 or the working electrode 32 with an unillustrated liquid electrolyte. The two-electrode cell shown in FIG. 22 includes a solid electrolyte 35 that electrically connects the working electrode 32 and the counter electrode 33. In FIG. 22, the solid electrolyte 35 covers each one part of the working electrode 32 and the counter electrode 33, but the solid electrolyte 35 may cover the whole of the base material 31, and preferably covers other portions than a portion to be attached to the skin. The solid electrolyte 35 is formed as a polymer electrolyte film or an electrolyte film obtained by impregnating a cellulose nanofiber film with an electrolyte. Due to the above electrolyte film being used as an electrolyte, when the two-electrode cell is attached to the skin and used, even in the case where the skin is not sweating, the solid electrolyte 35 functions as an electrolyte due to the moisture in the air, and can electrically connect the working electrode 32 and the counter electrode 33.

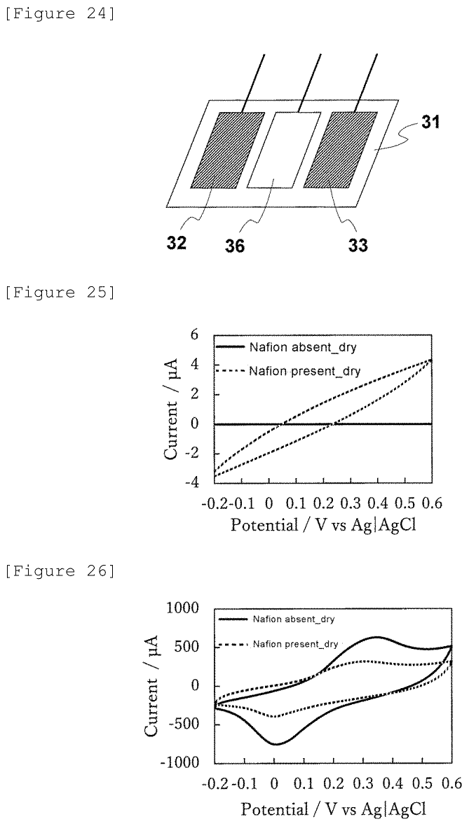

The three-electrode cells shown in FIG. 23 and FIG. 24 include a base material 31, and a working electrode 32, a counter electrode 33 and a reference electrode 36 which are installed on the base material 31. In the three-electrode cells shown in FIG. 23 and FIG. 24, the working electrode 32 is an electrode formed of the above composite film. In addition, the three-electrode cells shown in FIG. 23 and FIG. 24 may have a solid electrolyte 35 that electrically connects the working electrode 32, the counter electrode 33 and the reference electrode 36. When the solid electrolyte 35 is not used, the electrode is used by impregnating, for example, the base material 31 or the working electrode 32 with a liquid electrolyte. It is preferable for the solid electrolyte 35 to cover each one part of the working electrode 32, the counter electrode 33 and the reference electrode 36, but is also acceptable to cover the whole of the base material 31, and is more preferable to cover other portions than a portion attached to the skin. Solid electrolyte 35 is formed of the above electrolyte film. Due to the above electrolyte film being used as an electrolyte, when the three-electrode cell is attached to the skin and used, even in the case where the skin is not sweating, the solid electrolyte 35 functions as an electrolyte due to the moisture in the air, and can electrically connect the working electrode 32 and the counter electrode 33.

In addition, examples of other electrochemical cell devices include a biofuel cell. In the biofuel cell, the composite film is attached, for example, to a current collector such as a metal film, and is used. The composite film is provided with gaps, and accordingly can immobilize, for example, a molecular recognition body thereon. In the present embodiment, the molecular recognition body refers to a substance which exhibits affinity, selectivity or the like for a specific molecule or a molecule having a specific molecular structure in a part thereof, by an intermolecular interaction, for example, such as a hydrogen bond, a hydrophobic bond, and the van der Waals force. Examples of the molecular recognition body include enzymes and microorganisms. The enzyme or microorganism immobilized on the composite film reacts with a substance in a liquid which has been absorbed in the gap of the composite film. For example, when the substance in the liquid is oxidized or reduced by the enzyme or the microorganism, an electron is generated in the composite film. Thereby, the biofuel cell can generate an electric current. In addition, the composite film has a large surface area, accordingly has a wide reaction region between the enzyme or the microorganism and the substance in the liquid, and can efficiently generate an electric current. Furthermore, the composite film has flexibility, and can be used in close contact with the body to be contacted; and accordingly, the biofuel cell can be used in a state of being attached to the skin of the living body. When the composite film is attached to the skin of the living body, sweat or the like which has oozed from the skin of the living body is absorbed by the gap of the composite film. The biofuel cell causes an enzymatic reaction with the use of a substrate contained in sweat, and can generate an electric current. For example, when lactic acid dehydrogenase is immobilized on the composite film, lactic acid in sweat reacts with the lactic acid dehydrogenase in the composite film, and generates an electron. Thereby, the movement of electrons occurs in the composite film, and the biofuel cell can generate an electric current. For information, the composite film can employ a molecular recognition body corresponding to a target substance such as an antibody, DNA or RNA containing an aptamer, an artificial antibody formed from a molecular imprint polymer or an ion-selective molecule, in place of the enzyme. When the target substance is a redox body, the concentration of the target bound to the molecular recognition body can be quantified from the current response in the composite film. When the target substance bound to the molecular recognition body is not oxidized and reduced, the concentration of the target can be electrochemically quantified by a change in potential difference or impedance in the composite film. On the other hand, when the above target substance exists in a certain amount, enzymes, microorganisms, antibodies, and DNA or RNA containing aptamers which have adsorbed to the composite film can be electrochemically quantified with the use of these mechanisms.

(Method for Producing Composite Film)

The composite film can be obtained, for example, by the following method. When a gold nanoparticle is used as the electroconductive nanoparticle, first, a dispersion liquid of gold nanoparticles and a dispersion liquid of cellulose nanofibers are mixed, and a mixed dispersion liquid of gold nanoparticles and cellulose nanofibers is obtained. The cellulose nanofiber is well dispersed in water. Because of this, the cellulose nanofibers are easily mixed with the gold nanoparticles in water serving as a medium. In this mixed dispersion liquid, the gold nanoparticle and the cellulose nanofiber are spontaneously bonded by hydrogen bonding. The obtained mixed dispersion liquid is subjected to a molding process by a method such as suction filtration, and dried, and thereby a composite film can be produced. A known apparatus can be used for drying, and the condition is not particularly limited as long as the composite film is formed without being altered under the condition, and is usually at a temperature of 5 to 40° C. in the atmosphere. In addition, the composite film can be produced in the same way also in the case where another electroconductive nanoparticle than the gold nanoparticle is used.

In addition, it is also acceptable to obtain a composite film by immersing a cellulose nanofiber film or a solid material having a cellulose nanofiber film formed on the surface thereof, in a dispersion liquid of the electroconductive nanoparticle, and adding the electroconductive nanoparticle to the cellulose nanofiber film. In this case, examples of cellulose nanofiber film include a sheet-shaped cellulose nanofiber film and a cellulose fiber structure. The sheet-shaped cellulose nanofiber film can be produced, for example, by molding a solution containing a cellulose nanofiber film obtained by bacteria synthesis or containing cellulose nanofibers, by a method such as suction filtration. The immersion condition may be appropriately set depending on an application and a required function of the composite planar body to be obtained, but is usually 0.5 to 120 hours in the dispersion liquid of which the liquid temperature is 5 to 40° C. In addition, it is preferable to stir the dispersion liquid, because the electroconductive nanoparticles can be added (precipitated) to the cellulose nanofiber film so as to exist in such a state that the nanoparticles are dispersed in the film. Thereby, the electroconductive nanoparticles can add effects of enhancement of the electrical conductivity and physical strength, and the like, to the cellulose nanofiber film.

The dispersion liquid of gold nanoparticles can be prepared as an aqueous solution that includes a metal compound containing gold, and optionally a binder. Examples of the metal compound include hydrogen tetrachloroaurate (III) tetrahydrate, chloroauric (I) acid, and gold chloride (III). Examples of the binder include: citric acid, sodium citrate, ascorbic acid, sodium ascorbate, potassium carbonate, ammonia, methanol, and ethanol; derivatives of aniline, pyrrole and thiophene, and polymers thereof; and molecules having an alkyl chain or a benzene ring, and molecules having a thiol group, a disulfide group, an amino group, an imino group, a carboxy group or a carbonyl group, at a terminal or both terminals of any of the molecules. The binder may be appropriately set depending on the application and required function of the composite film to be obtained, but when a sulfur compound is added as a binder, the content of the sulfur compound per 1 g of the composite film can be preferably adjusted to 100 μg or less, and more preferably 10 μg or less. In addition, when a binder is not used and when a binder containing no sulfur is used, a sulfur-free composite film can be obtained.

The concentration of the metal compound in the dispersion liquid is approximately 1×10⁻⁵ to 1×10⁻¹ mass % in terms of metal. In addition, the mass ratio of the cellulose nanofiber to the metal in the metal compound is approximately 1:0.1 to 3.

The method for producing the composite film may further include a step of hot-pressing the composite film. The hot pressing can be performed with the use of a known apparatus, and the set temperature, pressure and time period can be appropriately set depending on the application and required function. Specifically, because the heat-resistant temperature of the cellulose nanofiber is approximately 350° C., the sheet-shaped composite film of the electroconductive nanoparticle/cellulose nanofiber is hot-pressed at 100° C. to 350° C. and 10 MPa to 40 MPa. In addition, the processing time is approximately 1 to 10 minutes. In the case where the electroconductive nanoparticle is the metal nanoparticle, the surface of the composite film becomes smooth and the metallic glossiness (reflectance) is enhanced by the hot press; the filling rate and contact rate of the metal nanoparticles increase, and a network is formed in which an electroconductive path formed of a plurality of connected metal nanoparticles spreads in a planar shape; and accordingly, the electroconductivity becomes equivalent to the specific resistivity of the pure metal of the metal nanoparticle, though depending on the metal content. For example, even if the amount of gold used in a conventional gold foil is reduced to 20% or less by volume occupancy, a composite film having high electrical conductivity can be formed.

When the electroconductive nanoparticle is the metal nanoparticle, the method for producing the composite film may further include the step of further growing metal nanoparticles in the composite film. The step can be carried out by charging the composite film on which metal nanoparticles are immobilized, into a dispersion liquid containing metal nanoparticles, and stirring the resultant dispersion liquid. Thereby, the metal nanoparticle in the dispersion liquid adhere to the surface of the metal nanoparticle in the composite film, and thereby can grow the metal nanoparticle. Alternatively, a metal salt or a metal complex in the above dispersion liquid containing the metal nanoparticles is reduced and precipitated due to the metal nanoparticle in the composite film serving as a nucleus, and thereby can grow the metal nanoparticle.

The method for producing the composite film may include also a step (cleaning step) of cleaning the inside of the composite film with ultrapure water, after the composite film has been formed. Thereby, an excess salt and the like can be eliminated which have entered the gap between the nanofibers in the composite film, in the production or the like. In addition, the method for producing the composite film will be described in detail in Example 1. A body fat percentage measuring device and a sensor element which use the composite film will be described below.

[Body Fat Percentage Measuring Device]

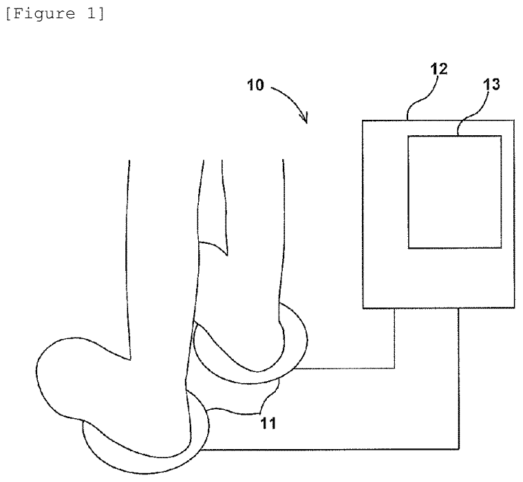

FIG. 1 shows a schematic view of the body fat percentage measuring device according to one embodiment of the present invention. As is shown in FIG. 1, the body fat percentage measuring device 10 includes a composite film 11, an AC power supply device 12, and an impedance measuring unit 13. The impedance measuring unit 13 is built in the AC power supply device 12. For information, the impedance measuring unit 13 may be configured to be independent of the AC power supply device 12. The composite films 11 are connected to the AC power supply device 12 and the impedance measuring unit 13, respectively. The AC power supply device 12 applies a sinusoidal current having a frequency of 50 kHz and a current value of 1.0 mA, to the composite film 11. The impedance measuring unit 13 detects the impedance in the composite film 11.

In the body fat percentage measuring device 10, the composite films 11 are used as an electrode. The composite films 11 are used in a state of being attached to the skin and in close contact. In this specification, “close contact” means a state in which at least the composite film is in a state of coming in close contact with a part of the body to be contacted, and the whole of the composite film 11 does not necessarily need to be in close contact with the body to be contacted. The subject, for example, attaches the composite film 11 to both heels, and measures a body fat percentage in a posture upright on the ground. For information, the place to which the composite film 11 is attached is not limited to both heels, but the composite film 11 may also be attached to the palm or the like, for example.

It is preferable to calculate the body fat percentage by a bioimpedance method, from the viewpoint of convenience and rapidness. The bioimpedance method is a method of passing a weak current through the body, measuring a resistance value at that time, and thereby assuming the body composition. The tissue that contains a large amount of electrolytes such as water in the living body such as the muscle has the property of conducting electricity well, and on the contrary, a fat component has the property of not conducting electricity easily. Because of this, as the fat content in the living body increases, the resistance value of the body rises. The bioimpedance method uses this property and calculates the body fat percentage.

Firstly, the body density (BD) is calculated from a height (Ht), a weight (W) and the obtained impedance (BI) of the subject, with the use of the following expression (1). Next, the obtained body density (BD) is substituted into Brozek's expression (expression (2)), and the body fat percentage is determined.

BD[g·cm⁻³]=1.1278−0.115×W×BI/Ht²+0.000095·BI expression (1)

body fat percentage [%]=(4.971/BD−4.519)×100 expression (2)

(W: body weight [kg], BI: impedance [Ω], and Ht: height [cm])

The body fat percentage measuring device 10 has the composite film 11. The composite film 11 can be in close contact with the body of the subject. In addition, as will be described in the Examples, a dielectric constant of the composite film 11 does not change by changes in the moisture content and the shape. Because of this, it is assumed that the body fat percentage measuring device 10 can measure the body fat percentage more accurately than conventional products.

[Sensor Element]

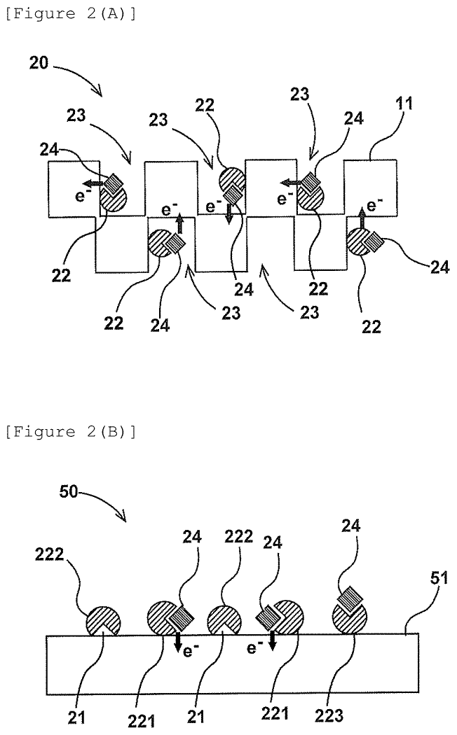

FIG. 2(A) shows a schematic cross-sectional view of a sensor element (hereinafter, also referred to as sensor element 20) according to one embodiment of the present invention. The sensor element 20 further includes a molecular recognition body 22 on the composite film 11. For information, the molecular recognition body 22 may be a holoenzyme containing a coenzyme. The molecular recognition body 22 has a size of approximately 5 to 30 nm. As is shown in FIG. 2(A), the molecular recognition body 22 is arranged in a plurality of gaps 23 of the composite film 11, in other words, in between the cellulose nanofibers. Though being not illustrated, the electroconductive nanoparticles exist on the surface including the gaps 23 of the composite film 11. The electroconductive nanoparticles add an effect of enhancing the electrical conductivity or the physical strength to the composite film 11, and the like. Furthermore, the molecular recognition bodies 22 are interspersed between the electroconductive nanoparticles.

In the case where the molecular recognition body 22 is the enzyme, the molecular recognition body 22 exhibits specific enzyme activity to a specific substance (hereinafter, also referred to as substrate 24). The sensor element 20 utilizes a chemical reaction that occurs by the catalysis of the molecular recognition body 22, which is an enzyme. For example, when the substrate 24 causes an oxidation reaction or a reduction reaction due to the molecular recognition body 22, an electron moves between the substrate 24 and the electroconductive nanoparticle of the composite film 11 directly via an unillustrated coenzyme, or indirectly via an unillustrated electron mediator. Electrons which correspond to the amount of the occurring chemical reaction move via the molecular recognition bodies 22 and the electroconductive nanoparticles in the composite film 11. Because of this, an electric current corresponding to the amount of the movement of the electrons is generated via the composite film 11. Specifically, the electric current is generated on the basis of the electrochemical reaction of the product which has been produced by the reaction between the molecular recognition body 22 and the substrate 24. Thereby, the enzyme sensor which uses the sensor element 20 can measure the amount of the substrate 24 that is contained in a sample which comes in contact with the sensor element 20.

It is preferable that the molecular recognition body 22 is, for example, an oxidase, a reductase, a dehydrogenase, or a holoenzyme containing a coenzyme. When the molecular recognition body 22 is the holoenzyme, the coenzyme is preferably flavin adenine dinucleotide, nicotine adenine dinucleotide, or pyrroloquinoline quinone. When the molecular recognition body 22 is the oxidase, the enzyme sensor which uses the sensor element 20 can measure the amount of the substrate 24 contained in the sample, by oxidizing the substrate 24. When the molecular recognition body 22 is the reductase, the enzyme sensor which uses the sensor element 20 can measure the amount of the substrate 24 contained in the sample, by reducing the substrate 24.

It is preferable that the oxidase is glucose oxidase or lactate oxidase. Thereby, the enzyme sensor which uses the sensor element 20 can measure, for example, the concentration of glucose or lactic acid which are contained in human sweat. For example, glucose oxidase produces gluconolactone and hydrogen peroxide, by an enzymatic reaction between glucose in the substrate and dissolved oxygen. The lactate oxidase causes an enzymatic reaction between lactic acid of the substrate and dissolved oxygen, and produces pyruvic acid and hydrogen peroxide. The hydrogen peroxide can be electrochemically detected with the use of an Ag|AgCl electrode, at +0.6 V. Because of this, the concentration of glucose or lactic acid can be quantified from the current response accompanying the generation of the hydrogen peroxide.

For information, as the molecular recognition body 22, a so-called holoenzyme may also be appropriately used which is a composite of an enzyme and a coenzyme. In addition, it is also acceptable to add an electron mediator molecule to the composite film 11, and immobilize the electron mediator molecule on the surface of the cellulose nanofiber or the electroconductive nanoparticle, with the use of a covalent bond or a bond via a thiol. The coenzyme or the electron mediator molecule has a function of transporting electrons by repeating a redox reaction in the molecule itself. Generally, flavin adenine dinucleotide, nicotine adenine dinucleotide or pyrroloquinoline quinone is used as the coenzyme. As the electron mediator, a substance having reversible redox characteristics can be used, such as hexacyano iron (III) ion, hexacyano iron (II) ion, a ferrocene derivative, or a quinone compound. Due to the action of the coenzyme or the electron mediator, the response and rapidity of the measurement can be enhanced, without being affected by dissolved oxygen.

In addition, the case has been described where an enzyme is used as the molecular recognition body 22, but the molecular recognition body 22 is not limited to the enzyme. The molecular recognition body 22 may also be, for example, an antibody that selectively binds to a target substance, DNA or RNA containing an aptamer, an artificial antibody formed from a molecular imprint polymer, an ion-selective molecule, or the like. When the target substance is a redox body, the concentration of the target bound to the molecular recognition body 22 can be quantified from the current response in the composite film 11. When the target substance bound to the molecular recognition body 22 is not oxidized and reduced, the concentration of the target can be electrochemically quantified by a change in potential difference or impedance in the composite film 11.

FIG. 2(B) is a schematic cross-sectional view of a conventional sensor element 50 in which a molecular recognition body 22 is immobilized on a flat plate electrode 51. In the case where the molecular recognition body 22 is an enzyme, when the molecular recognition bodies 22, which are the enzyme, are immobilized on the flat plate electrode 51, the molecular recognition bodies 22 are immobilized so as to point various directions on the flat plate electrode 51, as shown in FIG. 2(B). The enzyme has a recognition portion 21 that recognizes the substrate. As in the molecular recognition body 221 shown in FIG. 2(B), when the recognition portion 21 side is immobilized so as to come to a position relatively close to the flat plate electrode 51 without being covered with the flat plate electrode 51, electrons are smoothly transmitted to the flat plate electrode 51, in the case where electron movement has occurred in the recognition portion 21. However, as in the molecular recognition body 222 shown in FIG. 2(B), when the recognition portion 21 side is immobilized so as to be covered with the flat plate electrode 51, the recognition portion 21 side cannot recognize the target substance. In addition, as in the molecular recognition body 223 shown in FIG. 2(B), when the recognition portion 21 side is immobilized in a state where the recognition portion 21 side faces the opposite side to the flat plate electrode 51, the distance between the recognition portion 21 side and the flat plate electrode 51 becomes long. Because of this, even if the movement of an electron occurred in the recognition portion 21, there occurs a case where the electron is not transmitted to the flat plate electrode 51. Accordingly, in such a sensor element 50 that the molecular recognition body 22 is immobilized on the flat plate electrode 51, the molecular recognition body 22 may not be sufficiently utilized or the movement of an electron may not be detected, depending on the orientations of the molecular recognition bodies 22.

In contrast to this, the composite film 11 has various gaps 23 that communicate with the outside. The molecular recognition body 22 is arranged in the gap 23. The wall surface of the composite film 11 that forms the gap 23 thereon has a complicated shape compared to a plane. Because of this, even if the molecular recognition bodies 22 have various orientations with respect to the composite film 11, the recognition portion 21 sides are immobilized on positions relatively close to the composite film 11, without being covered with the composite film 11, in many cases, as shown in FIG. 2(A). Accordingly, the sensor element 20 can sufficiently utilize the molecular recognition body 22 regardless of the orientations of the molecular recognition bodies 22, and can smoothly detect the movement of electrons. Because of this, even when the amount of the substrate contained in the sample is a trace amount, it is assumed that the enzyme sensor which uses the sensor element 20 can measure the substrate with high accuracy, because of causing a reaction with high efficiency as compared with an enzyme sensor which uses the flat plate electrode 51. For example, even when the enzyme sensor which uses the sensor element 20 is used as a wearable measuring device and is used in close contact with the living body, the sensor element 20 can measure the concentration or the like of a trace amount of glucose or lactic acid contained in the human sweat. In addition, the composite film 11 has a structure having a large number of gaps 23, and accordingly, sweat permeates into the gaps 23. Because of this, the composite film 11 can efficiently measure a trace amount of glucose or lactic acid in a solution.

The sensor element 20 is obtained by immobilizing the molecular recognition body 22 on the composite film 11 by a known method. For example, the molecular recognition body 22 is immobilized on the cellulose nanofiber in the composite film 11, by covalent bonding, bonding via a thiol, or electrostatic interaction. For information, the sensor element 20 may include a single molecular recognition body 22, or may also include a plurality of molecular recognition bodies 22. In addition, a method for manufacturing the sensor element will be described in detail in Example 10 and Example 16.

### EXAMPLES

The present invention will be described in more detail with reference to Examples below, but the present invention is not limited to these Examples, and modifications and improvements within the scope in which the object of the present invention can be achieved are included in the present invention. For information, the ultrapure water which was used in the present Example was filtered, then adjusted in pH, passed through a reverse osmosis membrane and an ion exchange membrane, and subjected to ultraviolet sterilization treatment. All reagents which were used in the present Example were special grade reagents, and unless otherwise specified, reagents produced by FUJIFILM Wako Pure Chemical Industries, Ltd. were used.

### Preparation Example 1

<Preparation of Dispersion Liquid of Gold Nanoparticle>

To 400 mL of ultrapure water, 12 mL of a 1 wt. % aqueous solution of gold chloride (III) acid tetrachloride and 9 mL of a 2 wt. % aqueous solution of sodium citrate were added, the mixture was stirred at 80° C. for 20 minutes with the use of a stirrer, and a dispersion liquid of gold nanoparticles (average particle diameter: 30 nm, and 0.0136 wt. %) was obtained.

### Example 1

<Preparation of Composite Film>

To 0.5 g of a 2 mass % solution of cellulose (biomass nanofiber BiNFi-s IMa-10002, produced by Sugino Machine Limited), 250 mL of the above dispersion liquid of gold nanoparticles was added, the mixture was stirred at room temperature with the use of a stirrer for 1 minute, and a mixed dispersion liquid of gold nanoparticle/cellulose nanofiber was obtained. (Hereinafter, gold nanoparticle/cellulose nanofiber is also referred to as AuNP/CNF.) The mixed dispersion liquid of AuNP/CNF was subjected to suction filtration for 5 minutes by a suction filtration device (manufactured by Merck Millipore Corporation) in which a membrane filter (Omnipore Membrane Filter, pore size of 1 μm, and manufactured by Merck Millipore Corporation) made from PTFE was set, and a mixture of AuNP/CNF was precipitated on the membrane filter. The AuNP/CNF mixture was taken out together with the membrane filter, was placed on a hot plate (C-MAG HP10, manufactured by IKA), was heated at 130° C. for 2 minutes to be dried; and then the composite film (hereinafter, also referred to as AuNP/CNF film) was peeled from the membrane filter, and an AuNP/CNF film (gold: 13 vol. %) was obtained. The obtained AuNP/CNF film had self-standing properties and flexibility. For information, the filtrate obtained by filtration was colorless and transparent. Accordingly, it is assumed that all gold nanoparticles in the dispersion liquid of gold nanoparticles remain in the AuNP/CNF mixture on the membrane filter.

### Example 2

<Surface Observation of AuNP/CNF Film>

The surface of the AuNP/CNF film (gold: 13 vol. %) was observed with the use of a scanning electron microscope (SEM, Miniscope®, TM 3030, manufactured by Hitachi High-Tech Corporation). A photograph of the top surface is shown in FIG. 3.

From FIG. 3, it was confirmed that the AuNP/CNF film had a large number of gaps. In addition, white particles each having a diameter of approximately 30 nm shown in FIG. 3 are the gold nanoparticles, and thread-like fibers each having a diameter of approximately 100 nm are the cellulose nanofibers. It is considered that a hydrogen bond is formed by a carboxy group of citric acid, which is a protecting group of the gold nanoparticle, and a hydroxy group of cellulose, and the gold nanoparticle adheres to the cellulose nanofiber.

### Example 3

<Measurement of Moisture Content and Resistance Value of AuNP/CNF Film>

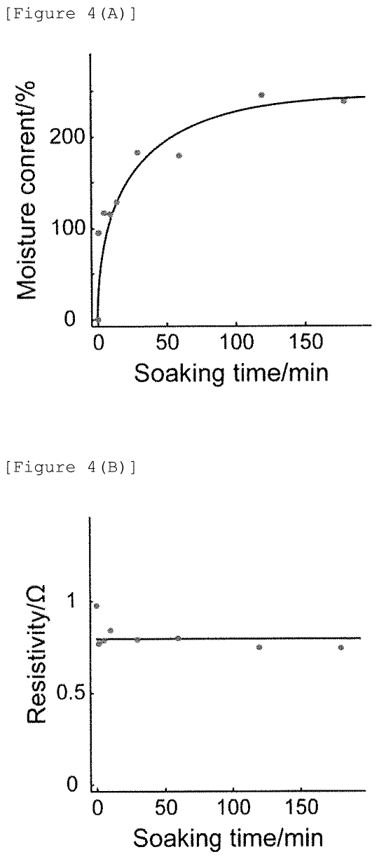

The AuNP/CNF film (gold: 13 vol. %) was immersed in ultrapure water for a predetermined time. The AuNP/CNF film was weighed before and after the immersion, and the resistance value was measured with a digital multimeter (34410A, manufactured by Agilent Technologies Japan Ltd., and applied current: 1 mA). The results are shown in FIG. 4(A) and FIG. 4(B). FIG. 4(A) shows a graph showing a change with time of the moisture content when the AuNP/CNF film is immersed in water, and FIG. 4(B) shows a graph showing a relationship between the elapsed time and the resistance value when the AuNP/CNF film is immersed in water.

As shown in FIG. 4(A), a weight of the AuNP/CNF film became 2.5 times greater at 120 minutes after immersion in ultrapure water than immediately after immersion, and after that, a significant change was not observed. In addition, as shown in FIG. 4(B), the resistance value of the sample at each time point showed a low value of 1Ω or smaller, regardless of the moisture content, and the fluctuation of the measured values was 0.5Ω or smaller. In other words, in the AuNP/CNF film (gold: 13 vol. %), the change in the resistance value caused by the increase or decrease of the liquid existing in the plurality of gaps is 0.5Ω or smaller. Furthermore, after this evaluation experiment, the AuNP/CNF film was sufficiently dried, and the resistance value was measured again; and as a result, the resistance value showed 0.76Ω. From the results, it was confirmed that the AuNP/CNF film had no influence on the resistance value even when having contained water, maintained high electrical conductivity even in a solution, and could be used as an electrode.

### Example 4

<Resistance Value and Tensile Strength with Respect to Gold Concentration Contained in AuNP/CNF Film>

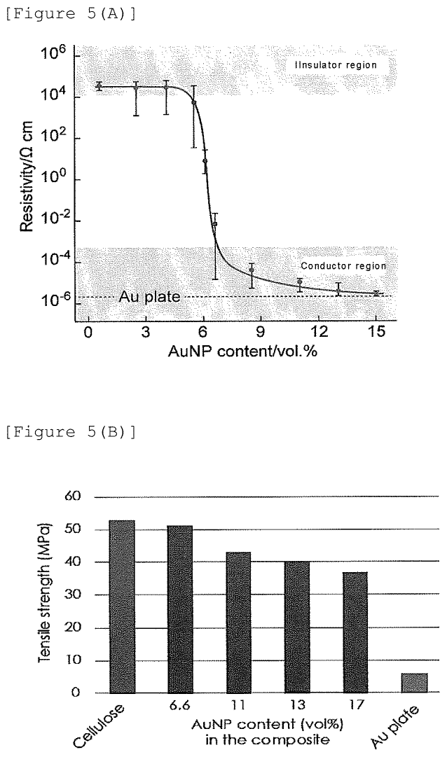

A plurality of AuNP/CNF films having different gold concentrations were prepared in the same way as in Example 1, except that only the gold concentration was changed in the preparation example of the dispersion liquid of gold nanoparticles. The specific resistance value of the AuNP/CNF film was measured with a digital multimeter (34410 A, manufactured by Agilent Technologies Japan Ltd., and applied current: 1 mA) and an ultrahigh resistance/microampere meter (8340A, manufactured by ADC Corporation). The results are shown in FIG. 5(A). In addition, the tensile strengths of the AuNP/CNF film (6.6 vol. %, 11.0 vol. %, 13.0 vol. %, and 17.0 vol. %), the CNF film and a gold foil were measured. The results are shown in FIG. 5(B). For information, the tensile strengths of the AuNP/CNF film and the CNF film were measured at 25° C. with the use of a digital force gauge (FJGN-50, manufactured by Nidec-Shimpo Corporation), after each film was cut into 2×2 cm. A gold foil (99.95%, AU-173174, manufactured by Nilaco Corporation) was measured in the same manner. The thickness of the AuNP/CNF film was measured with the use of a scanning electron microscope (SEM, TM 3030, manufactured by Hitachi High-Tech Corporation).

As is shown in FIG. 5(A), the specific resistivity of the AuNP/CNF film gradually decreased as the gold content in the film increased, and rapidly decreased when the gold content was 6 vol %. In addition, when the gold content of the AuNP/CNF film became 13 vol %, the specific resistivity exhibited equivalent specific resistivity (2.9×10⁻⁶ Ωcm) to that of the gold plate, which indicated that the amount of gold to be used was significantly reduced. In addition, the AuNP/CNF film had the film thickness of 5 to 10 μm, and as shown in FIG. 5(B), exhibited a tensile strength of 5 times or more higher than that of the gold plate, though having been flexible.

### Example 5

<Production of Electrode Using AuNP/CNF Film>

AuNP/CNF films (that were AuNP/CNF films in which gold contents were 2.5 vol. % to 17.0 vol. % (2.5 vol. %, 3.8 vol. %, 5.7 vol. %, 6.4 vol. %, 6.6 vol. %, 11.0 vol. %, 13.0 vol. % and 17.0 vol. %), respectively, and were produced in the same way as in the preparation of the above composite film) were each cut into a circular shape having a diameter of 1 cm, and a part was sandwiched between tapes of Teflon®, which were each cut into a circular shape having a diameter of 6 mm. A gold wire was connected to the AuNP/CNF film as a lead wire, and an AuNP/CNF film electrode was obtained.

### Example 6

<Cyclic Voltammetry (CV) Measurement>

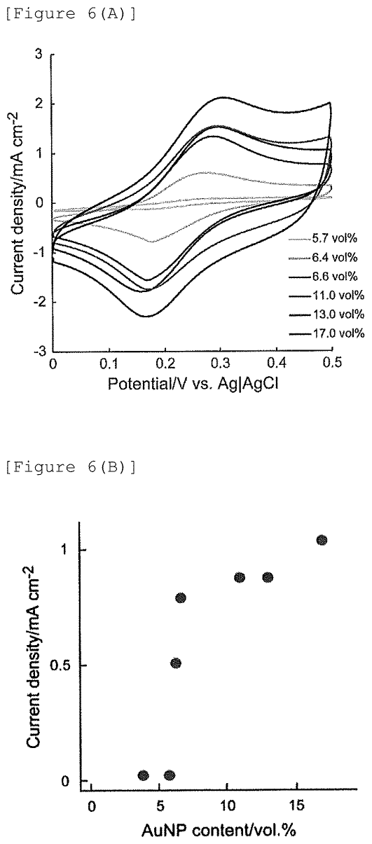

An aqueous solution of 0.1 M KCl and a solution of 5 mM K₃[Fe(CN)₆] which was prepared with an aqueous solution of 0.1 M KCl were prepared as electrolytic solutions. The AuNP/CNF film electrodes (gold: 3.8 vol. %, 5.7 vol. %, 6.4 vol. %, 6.6 vol. %, 11.0 vol. %, 13.0 vol. % and 17.0 vol. %) were each employed as a working electrode, an Ag|AgCl electrode was employed as a reference electrode, and a Pt coil electrode was employed as a counter electrode; and each electrode was immersed in an electrolytic solution. CV measurement was conducted with the use of cyclic voltammetry (ALS842B, manufactured by B.A.S Inc.) at a sweep rate of 50 mVs⁻¹. The results of the CV measurement in a solution of 5 mM K₃[Fe(CN)₆] are shown in FIG. 6(A). FIG. 6(B) is a graph in which the peak current values were plotted with respect to the volume occupancy of gold in the AuNP/CNF film. For information, in FIG. 6(A), a measurement value of the AuNP/CNF film electrode in which the gold concentration is 3.8 vol. % is not shown.

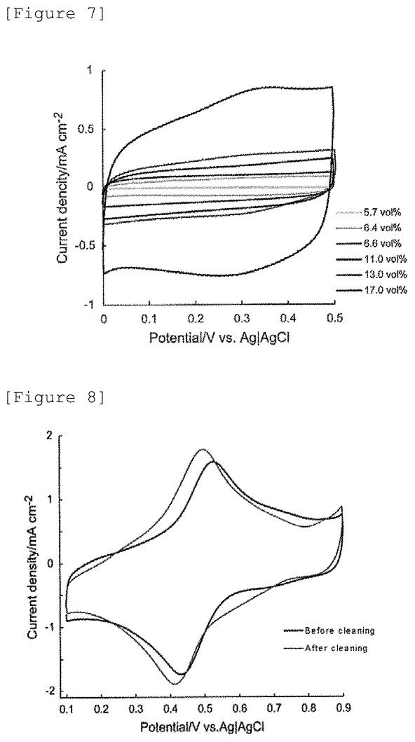

From the voltammogram shown in FIG. 6(A), a typical response based on a redox of ferricyanide was observed, when the gold content in the AuNP/CNF film was 6.4 vol. % or more. From FIG. 6(B), when the gold content in the AuNP/CNF film became 11 vol. % or more, a change in the peak current value became not to be observed. In addition, the results of CV measurement conducted in a solution of 0.1 M KCl are shown in FIG. 7. It was confirmed from FIG. 7 that the background increased as the amount of gold in the AuNP/CNF film increased.

### Example 7

<Influence of Cleaning Operation of AuNP/CNF Film>

In order to remove potential surface contaminants, the following operations were performed, and CV measurements before and after the operation were performed. An aqueous solution of 0.1 M H₂SO₄ was prepared as an electrolytic solution. An AuNP/CNF film electrode (gold: 13 vol. %) was employed as the working electrode, an Ag|AgCl electrode was employed as the reference electrode, and a Pt coil electrode was employed as the counter electrode, and each electrode was immersed in the electrolytic solution. Cyclic voltammetry (ALS842B, manufactured by B.A.S., Inc.) was used as a cleaning operation, and 100 cycles of CV measurement were carried out at a sweep rate of 200 mVs⁻¹. Before and after 100 cycles of the CV measurement, the CV was performed in an aqueous solution of 5 mM FeCl₃ which was dissolved in the aqueous solution of 0.1 M H₂SO₄. The sweep rate is 50 mVs⁻¹. The results are shown in FIG. 8.

As shown in FIG. 8, when voltammograms before and after cleaning were compared, the magnitude of the charging current almost did not change. Because of this, it is presumed that the change in the charging current is not caused by contamination of the AuNP/CNF film, but the structure of the AuNP/CNF film itself is a factor for generating the charging current.

### Example 8

<Evaluation of Chemical Resistance of AuNP/CNF Film>

In order to evaluate the chemical resistance of the AuNP/CNF film, the following operations were performed. The AuNP/CNF films were immersed in Falcon tubes filled with various treatment solutions, respectively. HCl (1 M), NaOH (1 M), ethanol, toluene and 5% neutral detergent were used as the various treatment solutions. The immersed AuNP/CNF film was treated with ultrasound (45 kHz) for 30 minutes. The specific resistivity of the AuNP/CNF film was measured with a digital multimeter (34410 A, manufactured by Agilent Technologies Japan Ltd., and applied current: 1 mA). With the use of the digital multimeter, the film was placed between a pair of electrodes (0.3 mm) having a gap of 3 mm, and the specific resistivity was calculated as an average value of the electric resistances which were measured three times at 25° C. Here, the film thickness was set to 50 nm. Absorption spectra of solutions before and after the treatment were measured and compared. Into an absorption spectrum measuring cell, 3 mL of each of solutions before and after the treatment was charged, and the absorption spectrum was measured with the use of an ultraviolet and visible spectrophotometer (V-750, by JASCO Corporation).

No change was observed in the specific resistivities of the AuNP/CNF films and in the absorption spectra of the solutions before and after treatment. Accordingly, it is assumed that there is no outflow of the gold nanoparticle to each chemical agent; and that the gold nanoparticle not only chemically adheres to but also is surrounded by cellulose nanofibers, and is in a state of resisting also being physically released.

### Example 9

<Evaluation of Electrochemical Properties of AuNP/CNF Film>

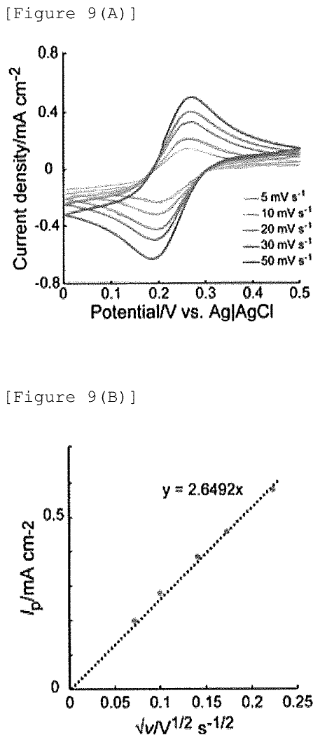

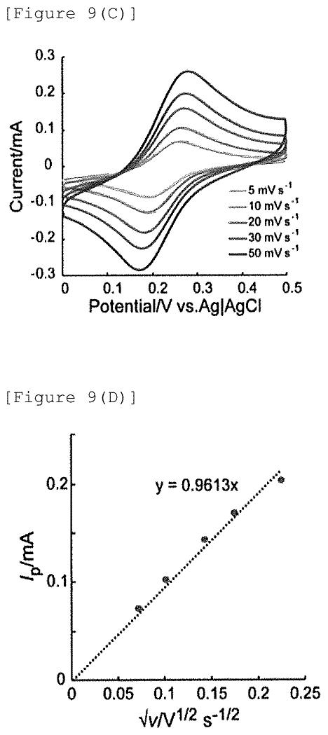

A solution of 5 mM K₃[Fe(CN)₆], which was prepared with an aqueous solution of 0.1 M KCl, was prepared as an electrolytic solution. An AuNP/CNF film electrode (gold: 13 vol. %) or a gold disk electrode was employed as the working electrode, an Ag|AgCl electrode was employed as the reference electrode, and a Pt coil electrode was employed as the counter electrode, and each electrode was immersed in the electrolytic solution. The AuNP/CNF film electrode was subjected to CV measurements with the use of cyclic voltammetry (ALS842B, manufactured by B.A.S Inc.) at some sweep rates of 5 to 50 mVs⁻¹, respectively. The results are shown in FIG. 9(A) and FIG. 9(B). FIG. 9(A) shows CV measurement results, which used an AuNP/CNF film electrode, and FIG. 9(B) shows a graph obtained by plotting peak current values with respect to a square root of the sweep rate, from the results of FIG. 9(A). FIG. 9(C) shows CV measurement results, which used the gold disk electrode, and FIG. 9(D) shows a graph obtained by plotting peak current values with respect to a square root of the sweep rate, from the results of FIG. 9(C).

In FIG. 9(A) and FIG. 9(C), the AuNP/CNF film electrode showed redox peaks similar to those of the gold disc electrode. In addition, the peak current value rose as the sweep rate increased. At this time, when the peak separation of each electrode was determined, the value was 73 mV for the AuNP/CNF film electrode, and was 64 mV for the gold disc electrode. Each value showed a value close to 57 mV which is a theoretical value of a one-electron transfer reaction, and it was confirmed that the reaction was a generally reversible reaction. In addition, when the peak current value was plotted with respect to the square root of the sweep rate, as shown in FIG. 9(B) and FIG. 9(D), the obtained line drew a straight line based on the theoretical expression (the following expression (3)) of the peak current, and it was confirmed that this system is a reversible process of the diffusion control.

In addition, when the diffusion coefficient D was calculated from a voltammogram which was obtained when the gold disk electrode was used as the working electrode, the value was 3.9×10⁻⁶ cm²/S. This value was substituted into the following expression (3), and the area of the AuNP/CNF film electrode was calculated, and was 0.36 cm². This value was approximately 1.3 times a geometrical area (0.28 cm²) of the AuNP/CNF film electrode. Accordingly, it is considered that the surface area increases due to the particle properties of a large number of gold nanoparticles which exist on the surface of the AuNP/CNF film.

Ip(peak current)=269n^{3/2}AD^{1/2}Cv^{1/2} (3)

(In the expression, n: reaction quantum number, A: electrode area cm², D: diffusion coefficient cm²/S, C: concentration mol/L, and v: sweep rate (V/s).)

### Reference Example 1

(Quantitative Determination of Glucose Concentration by Colorimetric Method)

Hereinafter, a colorimetric quantitative method of a glucose solution by a glucose oxidase/peroxidase (GOD/POD) method will be described as Reference Example.

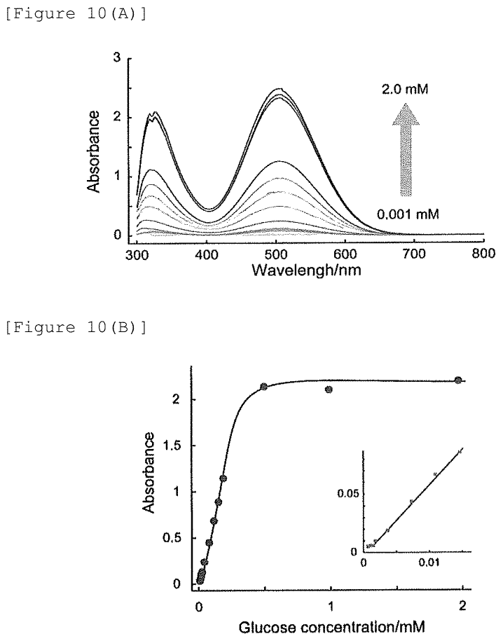

A staining reagent was prepared with the use of Lab Assay® glucose. As the staining reagent, 4-aminoantipiline and phenol were used. With 3 mL of the staining reagent, 20 μL of glucose having a predetermined concentration (2.8 mM to 0.3 M) was mixed, and the mixture was reacted in a thermostatic chamber (37° C.) for 5 minutes. Glucose oxidase enzymatically reacts glucose with dissolved oxygen, and produces gluconolactone and hydrogen peroxide. Aminoantipiline and phenol cause oxidative condensation reaction, in the presence of peroxidase and hydrogen peroxide, and form a red quinone dye. Into an absorption spectrum measuring cell, 3 mL of each solution after the reaction was charged, and the absorption spectrum was measured with the use of an ultraviolet and visible spectrophotometer (V-750, manufactured by JASCO Corporation). At this time, a color-developing reagent was used as a control, and the measurement range was set to 300 to 800 nm. The results are shown in FIG. 10(A). FIG. 10(B) shows a graph obtained by plotting the absorbances at 505 nm with respect to the glucose concentration in the measuring cell, on the basis of the results of FIG. 10(A). The figure in FIG. 10(B) shows a graph obtained by plotting the absorbances at 505 nm with respect to the concentration of the glucose added dropwise.

The absorbance of this red dye at 505 nm is proportional to the concentration of hydrogen peroxide. Because of this, the glucose concentration is quantified by the magnitude of the absorbance. As shown in FIG. 10(A), it was confirmed that the peak of the absorbance at 505 nm became larger as the glucose concentration increased. As shown in FIG. 10(B), it is understood that the glucose concentration can be quantified in a concentration range of 0.002 mM to 0.5 mM. As shown in FIG. 10(C), it is understood that the glucose concentration can be quantified in a concentration range of 0.3 mM to 75 mM. For information, a high concentration of glucose is quantified with the use of a calibration curve which is obtained from the graph of FIG. 10(C), but the sample needs to be diluted.

### Example 10

<Production of Glucose Sensor>

By a glutaraldehyde crosslinking method, 240 U/mg of glucose oxidase (hereinafter, also referred to as GOD) derived from Aspergillus niger was immobilized on the AuNP/CNF film electrode. Along with the immobilization, a mixed liquid of GOD, albumin derived from bovine serum (hereinafter, also referred to as BSA), and glutaraldehyde (hereinafter, also referred to as GA) was produced. A method for producing the mixed liquid is as follows.

(1) Approximately 5 mg of GOD was weighed out, and was dissolved in a 0.2 M phosphate buffer solution (pH 7.0) so as to become 12 U μL⁻¹, and a GOD solution was prepared.

(2) Approximately 11 mg of the BSA was weighed out, and dissolved in a 0.2 M phosphate buffer solution (pH 7.0) so as to become 110 mgmL⁻¹, and a BSA solution was prepared.

(3) A 25% GA liquid was dissolved in the 0.2 M phosphate buffer solution (pH 7.0) so as to become 7%, and a GA solution was prepared.

(4) Three μL of the GOD solution of (1), 29 μL of the BSA solution of (2), and 4 μL of the GA solution of (3) were mixed, and a mixed liquid in a total amount of 36 μL was obtained. Six μL (6 U) of the mixed liquid was added dropwise onto the AuNP/CNF film electrode (gold: 13 vol. %). The resultant AuNP/CNF film electrode was left under darkness for 24 hours, and a glucose sensor was obtained. After that, the produced glucose sensor was stored in the 0.2 M phosphate buffer solution (pH 7.0).

### Example 11

<Glucose Sensing Using AuNP/CNF Film Electrode in Bulk>

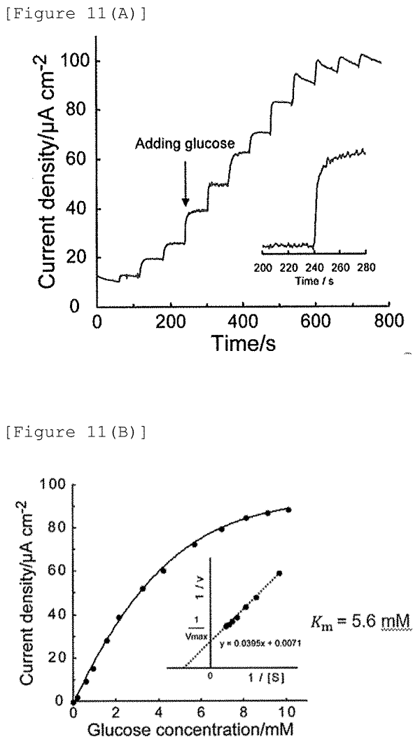

The above glucose sensor was used as the working electrode, and a platinum mesh electrode and an Ag|AgCl electrode were used as the counter electrode and the reference electrode, respectively, and amperometry (+0.6 V) was performed in 10 mL of a 0.2 M phosphate buffer solution (pH 7.0). A stirrer piece was charged into a cell, and the buffer solution was stirred at 500 rpm. After the electric current became stable, a solution of 25 mM glucose was added every minute, and a change in the electric current was measured. The results are shown in FIG. 11(A) and FIG. 11(B). FIG. 11(A) shows a graph of a current response at the time when the glucose solution has been added, and the figure in FIG. 11(A) is an enlarged graph of one part of FIG. 11(A). In addition, FIG. 11(B) shows a graph obtained by plotting current values with respect to a concentration on the basis of the results of FIG. 11(A), and the figure in FIG. 11(B) is a Lineweaver-Burk double reciprocal plot.

As shown in FIG. 11(A), when the glucose solution was added, the electric current rose quickly, and the electric current exhibited a stable response. This response is considered to have been able to be observed as the current response due to hydrogen peroxide which has been generated by the enzymatic reaction. In addition, as shown in FIG. 11(B), the current value rose as the glucose concentration increased, and the current response exhibited satisfactory concentration dependency in a glucose concentration region of 0.2 mM to 10 mM.

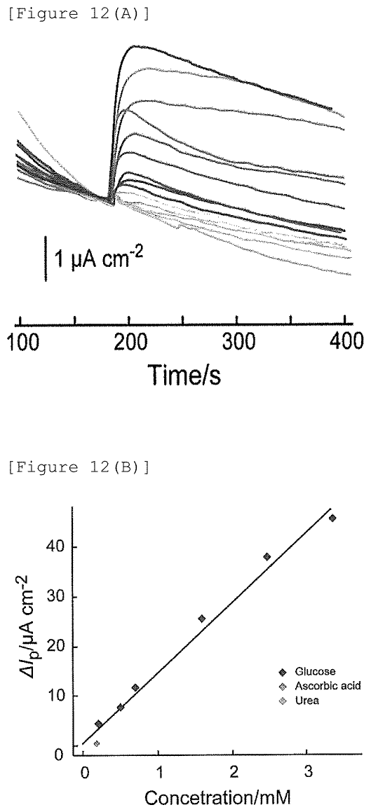

The glucose concentration at a lower concentration (0.001 mM to 0.1 mM) was further measured with the use of the glucose sensor using the AuNP/CNF film electrode, and the detection limit was examined. FIG. 12(A) shows a graph of a current response at the time when a glucose solution has been added, and FIG. 12(B) shows a graph obtained by plotting the current values with respect to a concentration on the basis of the results of FIG. 12(A). As shown in FIG. 12(A) and FIG. 12(B), it was confirmed that the lower limit at which the concentration could be measured was 0.01 mM, in the measurement which used the glucose sensor using the AuNP/CNF film electrode. Specifically, it was confirmed that the measurement method which used the glucose sensor using the AuNP/CNF film electrode could quantify a concentration range that was ten times wider than the colorimetric method. From the result, a wide concentration range can be quantified without diluting a sample, by using the glucose sensor using the AuNP/CNF film electrode, and application to glucose sensing in various situations can be expected.

In addition, as shown in FIG. 11(B), the current response with respect to the concentration indicated by the glucose sensor using the AuNP/CNF film electrode drew a curve based on the Michaelis-Menten equation shown by the following expression (4) which showed the enzyme reaction kinetics. The Michaelis-Menten constant Km, which represents the affinity between the enzyme and the substrate, was determined from analysis for the linear plot, and was calculated to be 5.6 mM. The Michaelis-Menten constant Km represents the affinity between the enzyme and the substrate, and when the constant is low, the affinity is regarded to be high. The value calculated this time is lower than the constant which was calculated with the use of the other glucose sensor (See: Z. Cao, Y. Zou, C. Xiang, Li-XianSun, and F. Xu, Anal. Letters, 2007, 40, 2116 and the like); and it was confirmed that the glucose sensor using the AuNP/CNF film electrode function as a sensor having high affinity. In addition, the porous structure of the AuNP/CNF film electrode enables a large electrochemically active surface and strong enzyme immobilization. It is assumed that the AuNP/CNF film has a large number of gaps and particle properties, thereby functions as a porous electrode, and exhibits high enzyme activity.

v=Vmax×[S]/(Km+[S]) Expression (4)

(In the expression, v: reaction rate, Vmax: maximum reaction rate, and [S]: concentration of substrate.)

### Example 12

<Evaluation of Selectivity of Glucose Sensor>

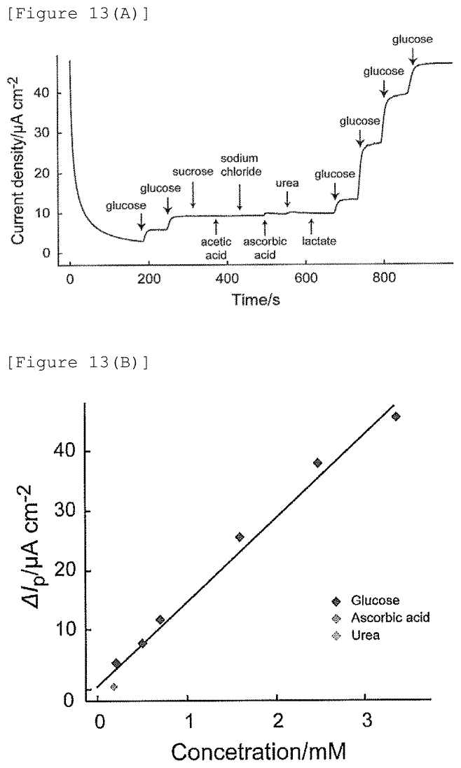

The above glucose sensor was used as the working electrode, and a platinum mesh electrode and an Ag|AgCl electrode were used as the counter electrode and the reference electrode, respectively, and amperometry (+0.6 V) was performed in 10 mL of a 0.2 M phosphate buffer solution (pH 7.0). A stirrer piece was charged into the cell, and the buffer solution was stirred at 500 rpm. After the electric current became stable, 100 μL of each of a solution of 25 mM glucose, a solution of 20 mM sucrose, a solution of 20 mM acetic acid, a solution of 20 mM sodium chloride, a solution of 20 mM ascorbic acid, a solution of 20 mM urea, a solution of 20 mM lactic acid, and a solution of 100 mM glucose was added every minute, and a change in the electric current was measured. The results are shown in FIG. 13(A) and FIG. 13(B). FIG. 13(A) shows a graph of a current response at the time when each solution has been added, and FIG. 13(B) shows a graph obtained by plotting current values with respect to glucose concentration on the basis of the results of FIG. 13(A).

As shown in FIG. 13(A) and FIG. 13(B), when interfering substances other than glucose were added, any change was not observed in the current response, and only when glucose was added, the current response occurred. It was confirmed that the AuNP/CNF film electrode having GOD immobilized thereon exhibited high selectivity for glucose. Sweat includes interference substances such as lactic acid, cortisol, sodium ion and chloride ion, in addition to glucose. Because of this, the glucose sensor using the AuNP/CNF film electrode can measure the glucose concentration without being hindered by the interfering substances contained in sweat.

### Example 13

<Sensing Glucose in Sweat>

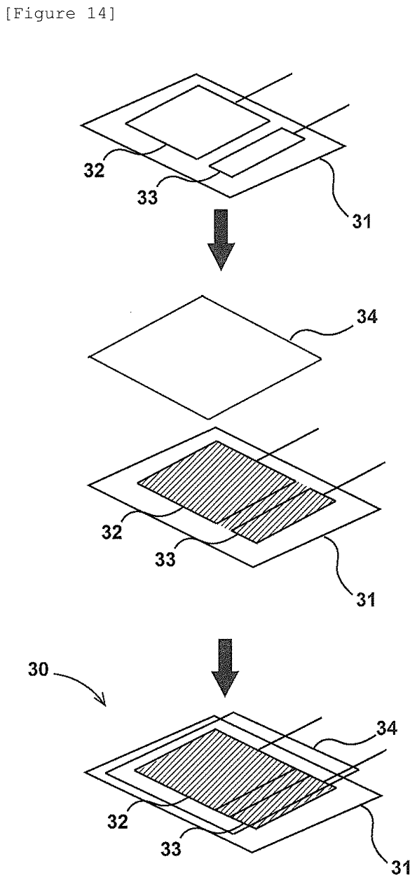

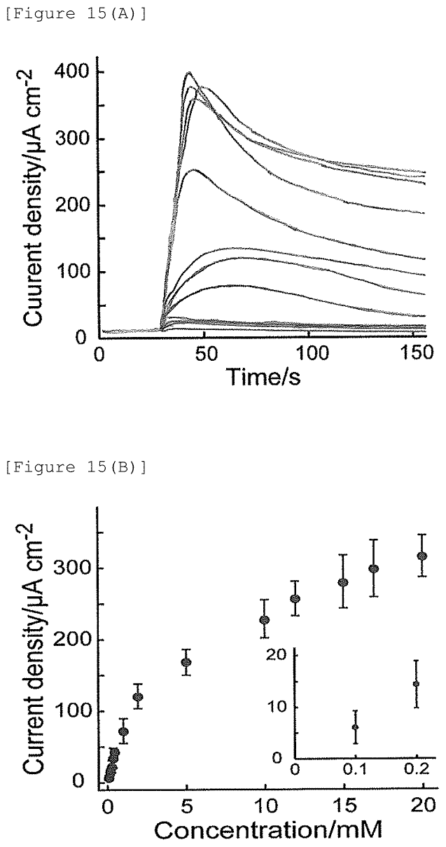

The AuNP/CNF film (gold: 13 vol. %) was cut out, and a two-electrode cell 30 as shown in FIG. 14 was produced. As shown in FIG. 14, a working electrode 32 (AuNP/CNF film electrode) and a counter electrode 33 were provided on a base material 31 at predetermined intervals. Subsequently, GOD was immobilized on the working electrode 32 in the same way as in the production of the above glucose sensor. Fifty μL of a phosphate buffer solution (pH 7.0) was added dropwise so as to cover both electrodes of the working electrode 32 and the counter electrode 33, and both the resultant electrodes were covered with a cover glass 34. Amperometry was performed at a constant potential of +0.6 V, and when the electric current became stable, a glucose solution was added. The results are shown in FIG. 15(A) and FIG. 15(B). FIG. 15(A) shows a graph of a current response at the time when a glucose solution has been added, and FIG. 15(B) shows a graph obtained by plotting the peak current values with respect to the concentration on the basis of the results of FIG. 15(A).

As shown in FIG. 15(A), a rise in the current value was confirmed along with an increase in glucose concentration. In addition, as shown in FIG. 15(B), the current response drew a curve based on the Michaelis-Menten equation, which showed satisfactory concentration dependency in a concentration region of 0.01 to 20 mM glucose. In the concentration region of 0.001 to 0.007 mM glucose, a current response corresponding to the concentration was not observed, and the detection limit of this glucose sensor was 0.01 mM. In addition, the Michaelis-Menten constants of the glucose sensors with the use of the AuNP/CNF films which were produced four times in the same way were all 5.0 to 5.6 mM. From this result, it was found that the glucose sensor of the two-electrode cell which used the AuNP/CNF film functioned as a glucose sensor having high affinity between the enzyme and the substrate, and had high reproducibility. In addition, when the glucose selectivity was evaluated, current values were plotted with respect to the concentration of glucose, and as a result, the current values showed the same values as in the measurement results in the bulk (the above glucose sensing using AuNP/CNF film electrode in the bulk), and it was confirmed that there was similarly reproducibility.

### Example 14

<Evaluation of Glucose Concentration in Sweat Due to Meal>

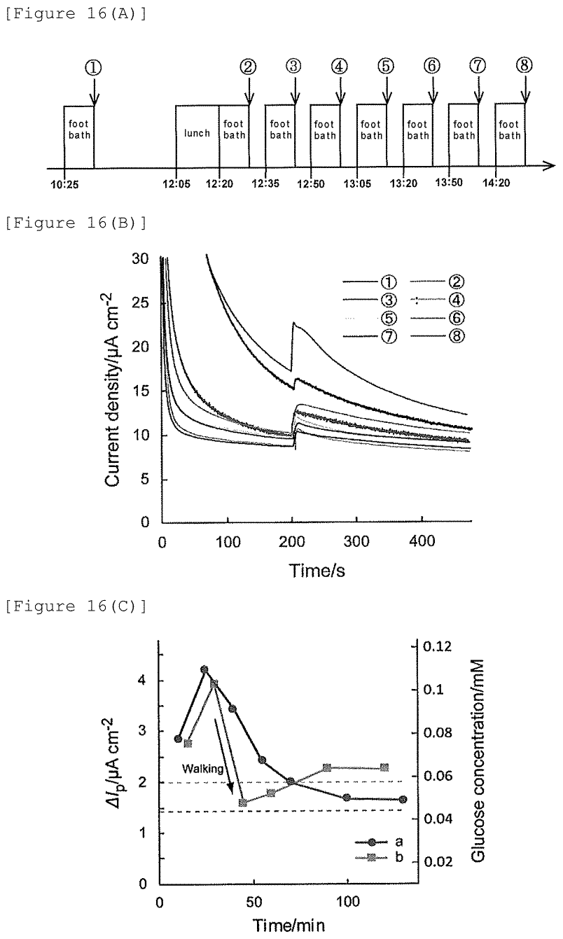

Sweat under normal conditions and sweats after a meal (0 to 120 minutes) were collected. The timings at which the sweats have been collected are shown in FIG. 16(A). For information, the blood sugar level depends on exercise intensity, and the glucose level decreases when the subject exercises so as to collect sweat. Accordingly, the subject stimulated sweating by a footbath, and the sweat was collected. With the use of the above glucose sensor, amperometry was performed at a constant potential of +0.6 V. When the electric current became stable, the collected sweat was added. With the use of the sweats before and after the meal, amperometry was performed, and the results are shown in FIG. 16(B). In addition, a broken line in FIG. 16(C) is a graph which indicates the current level of the sweat collected before the meal by a dotted line, and in which the current values are plotted with respect to elapsed time after the meal.

### Example 15

<Evaluation of Glucose Concentration in Sweats Depending on Exercise Intensity>

The subject walked after 30 minutes after the meal, at the time when the blood sugar level became highest, and the sweat was collected after 45 minutes after the meal. The glucose concentration was evaluated by the same operation as in the evaluation of the glucose concentration in the sweat due to the above meal. The result is shown as a graph of a broken line of b in FIG. 16(C).

As shown in FIG. 16(B) and FIG. 16(C), the current level showed an increase up to 25 minutes after the meal, and after 100 minutes, decreased to the original glucose level. In addition, when the subject exercised (walked) in between 30 minutes and 45 minutes after the meal, the current value largely decreased as compared with the time when the subject did not exercise. These changes in the current response coincided with blood glucose levels which were expected for healthy people. Accordingly, it was confirmed that the glucose sensor using the AuNP/CNF film electrode could accurately measure the glucose concentration contained in the sweat.

### Example 16

<Production of Lactic Acid Sensor>

Lactate oxidase (hereinafter, also referred to as LOD) derived from Aerococcus was immobilized on the AuNP/CNF film electrode by a glutaraldehyde crosslinking method. A mixed liquid of GOD, albumin derived from bovine serum (hereinafter, also referred to as BSA) and glutaraldehyde (hereinafter, also referred to as GA) was produced along with the immobilization. A method for producing the mixed liquid is as follows.

(1) LOD solutions were prepared by weighing out the LODs so as to become a predetermined concentration (14 UμL⁻¹), and dissolving the LODs in 0.2 M phosphate buffer solutions (pH 7.0), respectively.

(2) A BSA solution was prepared by weighing out approximately 11 mg of BSA, and dissolving the BSA in a 0.2 M phosphate buffer solution (pH 7.0) so as to become 110 mgmL⁻¹.

(3) A GA solution was prepared by dissolving a 25% GA solution in a 0.2 M phosphate buffer solution (pH 7.0) so as to become 7%.

(4) A mixed liquid in a total amount of 36 μL was obtained by mixing 3 μL of the LOD solution of (1), 29 μL of the BSA solution of (2), and 4 μL of the GA solution of (3). Onto the AuNP/CNF film electrode (gold: 13 vol. %), 6 μL (7 U) of the mixed liquid was added dropwise. The resultant electrode was left at rest under darkness for 24 hours, and a lactic acid sensor was obtained. After that, the produced lactic acid sensor was stored in a 0.2 M phosphate buffer solution (pH 7.0).

### Example 17

<Lactic Acid Sensing Using AuNP/CNF Film Electrode in Bulk>

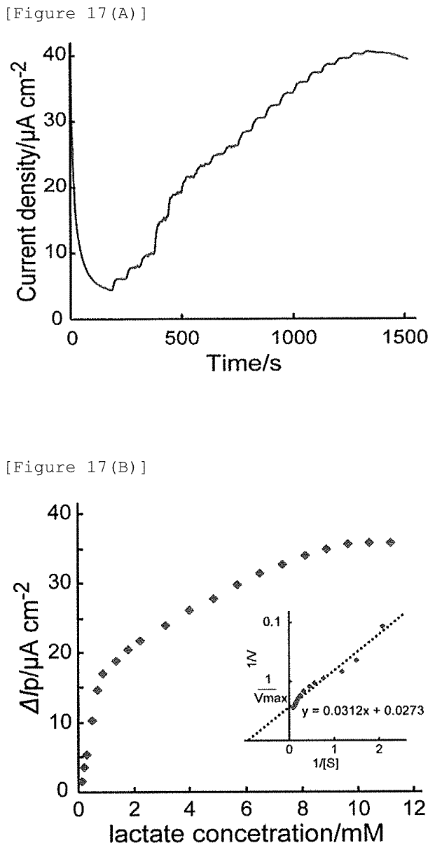

The above lactic acid sensor was used as the working electrode, and a platinum mesh electrode and an Ag|AgCl electrode were used as the counter electrode and the reference electrode, respectively, and amperometry (+0.6 V) was performed in 10 mL of a 0.2 M phosphate buffer solution (pH 7.0). A stirrer piece was charged into a cell, and the buffer solution was stirred at 500 rpm. After the electric current became stable, a solution of 25 mM lactic acid was added every minute, and a change in the electric current was measured. The results are shown in FIGS. 17(A) and 17(B). FIG. 17(A) shows a graph of a current response at the time when the lactic acid solution has been added, and the figure in FIG. 17(A) is an enlarged graph of one part of FIG. 17(A). In addition, FIG. 17(B) shows a graph obtained by plotting current values with respect to a concentration on the basis of the results of FIG. 17(A), and the figure in FIG. 17(B) is a Lineweaver-Burk double reciprocal plot.

As shown in FIG. 17(A), when the lactic acid solution was added, the current rose quickly, and the electric current exhibited a stable response. This response is considered to have been able to be observed as the current response due to hydrogen peroxide which has been generated by the enzymatic reaction. The lactate oxidase causes the enzymatic reaction between lactic acid and dissolved oxygen, and produces pyruvic acid and hydrogen peroxide. In addition, as shown in FIG. 17(B), the current value rose as the lactic acid concentration increased, and the current response exhibited satisfactory concentration dependency, in a lactic acid concentration region of 0.1 mM to 10 mM. The Michaelis-Menten constant Km was calculated to be 1.1 mM, from the analysis for the linear plot, and it was confirmed that the sensor functioned as a sensor having high affinity.

### Example 18

<Evaluation of Selectivity of Lactic Acid Sensor>

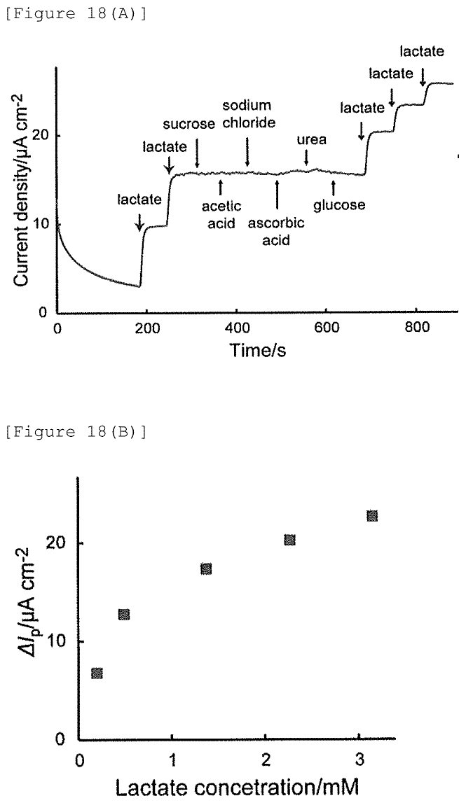

The above lactic acid sensor was used as the working electrode, and a platinum mesh electrode and an Ag|AgCl electrode were used as the counter electrode and the reference electrode, respectively, and amperometry (+0.6 V) was performed in 10 mL of a 0.2 M phosphate buffer solution (pH 7.0). A stirrer piece was charged into the cell, and the buffer solution was stirred at 500 rpm. After the electric current became stable, 100 μL of each of a solution of 25 mM lactic acid, a solution of 20 mM sucrose, a solution of 20 mM acetic acid, a solution of 20 mM sodium chloride, a solution of 20 mM ascorbic acid, a solution of 20 mM urea, a solution of 20 mM glucose and a solution of 100 mM lactic acid was added every minute, and a change in the electric current was measured. The results are shown in FIG. 18(A) and FIG. 18(B). FIG. 18(A) shows a graph of a current response at the time when each solution has been added, and FIG. 18(B) shows a graph obtained by plotting current values with respect to lactic acid concentration on the basis of the results of FIG. 18(A).

As shown in FIG. 18(A) and FIG. 18(B), when interfering substances other than the lactic acid were added, changes were not observed in current responses, and only when the lactic acid was added, the current response occurred. It was confirmed that the AuNP/CNF film electrode having LOD immobilized thereon exhibited high selectivity for the lactic acid. Because of this, the lactic acid sensor which uses the AuNP/CNF film electrode can measure the lactic acid concentration without being hindered by the interfering substances contained in sweat.

### Example 19

<Lactic Acid Sensing Using AuNP/CNF Film Electrode During Exercise>

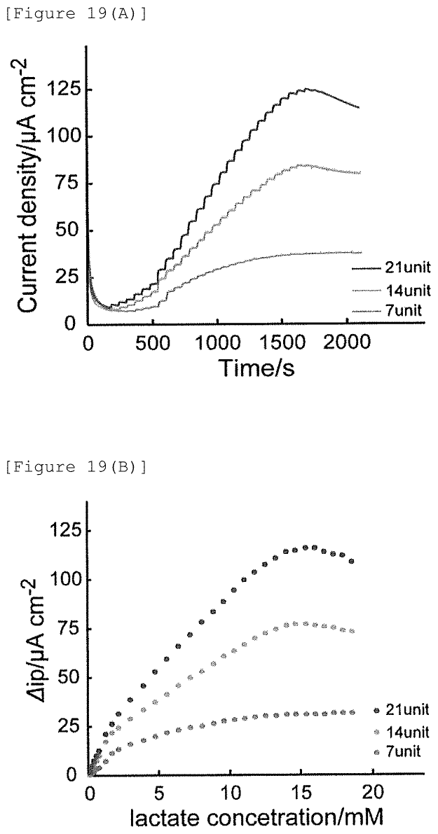

The concentration of lactic acid in sweat is 10 times or more the concentration of lactic acid in blood. In addition, the lactic acid concentration is said to increase depending on an increase in exercise intensity, and lactic acid content in the sweat of a healthy person increases from 4 to 25 mM during rest to 50 to 80 mM. For this reason, in order to measure the concentration of lactic acid in sweat without diluting the sweat, it is required that a sample having a concentration of at least 50 mM can be measured. For this reason, it is necessary to widen the measurable concentration range of the lactic acid sensor up to a high concentration. Then, the number of units of the LOD was increased, which was to be immobilized on the AuNP/CNF film electrode, and the current response accompanying the addition of lactic acid was evaluated. Specifically, in the production of the above lactic acid sensor, LOD solutions having three types of concentrations (14 UuL⁻¹, 28 UuL⁻¹ and 42 UuL⁻¹) were prepared. The numbers of units of the LOD immobilized on the AuNP/CN film electrodes were 7 U, 14 U and 21 U, respectively. The measurement was performed in the same way as in the above lactic acid sensing with the use of the AuNP/CNF film electrode in the bulk. The results are shown in FIG. 19(A) and FIG. 19(B). FIG. 19(A) shows a graph of a current response at the time when a lactic acid solution has been added. In addition, FIG. 19(B) shows a graph obtained by plotting the current values with respect to a concentration, on the basis of the results of FIG. 19(A).

As shown in FIG. 19(A) and FIG. 19(B), as the number of units of the LOD immobilized on the AuNP/CNF film electrode increased, the current value with respect to the lactate concentration increased. Thereby, it was confirmed that the upper limit of the measurable concentration range can be widened by increasing the number of units of the LOD immobilized on the AuNP/CNF film electrode. Accordingly, the lactic acid sensor using the AuNP/CNF film electrode can be used for monitoring the level of lactic acid in sweat.

### Example 20

<Change in Resistance Value Caused by Movement of the Human Body>

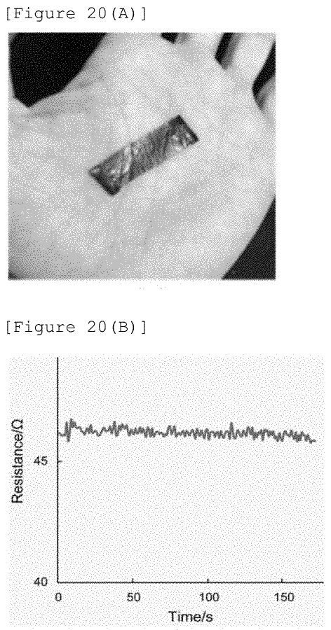

An AuNP/CNF film (gold: 13 vol. %) was wetted with a small amount of water, and then was attached to the palm. FIG. 20(A) shows a photograph in a state in which the AuNP/CNF film is attached to the palm. A gold wire was connected to the AuNP/CNF film, as a lead wire. The resistance value of the AuNP/CNF film, at the time when the palm was opened and closed every 1 second as the movement of the human body, was measured by a digital multimeter (34410 A, manufactured by Agilent Technologies Japan Ltd., and applied current: 1 mA). The result is shown in FIG. 20(B).

As shown in FIG. 20(A), it was confirmed that the AuNP/CNF film was attached to the skin when having been pressed against the palm. Furthermore, it was confirmed that the AuNP/CNF film came in contact with the skin along the asperities, that the AuNP/CNF film was not peeled off by the movement of the palm, and that the state of attaching to the palm was maintained. In addition, as shown in FIG. 20(B), the change in the resistance value due to the opening and closing of the hand was almost not confirmed, and was within an error range (2.0Ω or smaller) of the measured value, and stable electrical conductivity was maintained. From these results, it was confirmed that the AuNP/CNF film could flexibly correspond to the movement of the human body, and that the electrical conductivity with respect to the change in the state was also sufficiently stable without being affected by the movement of the human body.

### Example 21

<Measurement of Body Fat Percentage Using AuNP/CNF Film>

An AuNP/CNF film (gold: 13 vol. %) was attached to both heels of the subject, and the subjects (1 to 3) were in a state of standing upright on the ground. The AuNP/CNF film was connected to an AC power supply device (IM6, manufactured by Zahner-Elektrik GmbH & Co. KG), a sinusoidal current having a frequency of 50 kHz and a current value of 1.0 mA was applied thereto, and the impedance was measured. The body fat percentage was calculated according to the following calculation method, on the basis of the obtained impedance and the body length and body weight of the subject.

Body density (BD) was calculated from the body length (Ht) and body weight (W) of the subject and the obtained impedance (BI), with the use of the expression (5). Next, the obtained body density (BD) was substituted into Brozek's expression (expression (6)), and the body fat percentage was determined.

BD[g˜cm⁻³]=1.1278−0.115×W×BI/Ht²+0.000095·BI expression (5)

body fat percentage [%]=(4.971/BD−4.519)×100 expression (6)

(In the expression, W: body weight [kg], BI: impedance [Ω], and Ht: height [cm].)

In addition, the body fat percentage was measured with the use of a commercially available body composition analyzer (HBF-361, manufactured by OMRON Corporation), and the measured values were compared with the results obtained with the use of the AuNP/CNF film. The results are shown in Table 1. In the body fat percentages (%) shown in Table 1, A represents measurement results of body fat percentages with the use of the AuNP/CNF film of the present invention, and B represents measurement results of body fat percentages measured with a commercially available body composition analyzer.

TABLE 1

| | | | Body fat percentage (%) | |
|---|---|---|---|---|
| | | | | Results of |
| | | | Results of | measurement having |
| | | Body | measurement | used commercially |
| | Height | weight | having used | available body |
| | (cm) | (kG) | composite film | composition analyzer |
| 1 | 160 | 51 | 5.2 | 11.3 |
| 2 | 166 | 62 | 23.4 | 24.6 |
| 3 | 179 | 120 | 48.4 | 47.7 |

As shown in Table 1, the measurement results of the body fat percentage with the use of the AuNP/CNF film and the measurement results of the body fat percentage measured with the use of the commercially available body composition analyzer were compared, and as a result, it was confirmed that the same tendency was observed.

### Example 22

<Production of Three-Electrode Cell>

As shown in FIG. 24, a three-electrode cell was produced by employing an AuNP/CNF film electrode (gold: 13 vol. %) as a working electrode, an AgNP/CNF film electrode (silver: 20 vol. %) as a reference electrode, and an AuNP/CNF film electrode (gold: 13 vol. %) as a counter electrode, and then was subjected to cyclic voltammetry (CV) measurement at a sweep rate of 50 mVs⁻¹. The above three-electrode cell did not operate as an electrolytic cell in a dry state in which an electrolytic solution was not added, and the measurement was impossible. In the above three-electrode cell, the CNF film was immersed in a 5 wt. % Nafion™ (produced by Chemours Com.) solution, then was arranged so as to cover each part of the working electrode and the counter electrode, and the reference electrode, as shown in FIG. 22; and the electrodes were dried at 60° C. for 30 minutes, and a solid electrolyte film was formed. Even in the dry state in which the electrolytic solution was not added, the Nafion film contained a moisture equilibrium with the humidity of the atmosphere, and a polar group of Nafion is ionized and serves as the electrolyte; and accordingly, a current response to the voltage was observed. The results of the above CV measurements are shown in FIG. 25.

### Example 23

<Production of Three-Electrode Cell>

An aqueous solution of 0.1 M KCl containing 10 mM K₃[Fe(CN)₆] was prepared as an electrolytic solution. As shown in FIG. 24, 200 μL of an electrolytic solution was added dropwise so as to cover a three-electrode cell that was produced by employing an AuNP/CNF film electrode (gold: 13 vol. %) as a working electrode, an AgNP/CNF film electrode (silver: 20 vol. %) as a reference electrode, and an AuNP/CNF film electrode (gold: 13 vol. %) as a counter electrode, and the CV measurement was performed at a sweep rate of 50 mVs⁻¹. In addition, the CNF film was immersed in the 5 wt. % Nafion (produced by Chemours Com.) solution, then was arranged so as to cover each part of the working electrode and the counter electrode, and the reference electrode; and the electrodes were dried at 60° C. for 30 minutes, and a solid electrolyte film was formed. In the same way as described above, 200 μL of the electrolytic solution was added dropwise, and the CV measurement was performed at a sweep rate of 50 mVs⁻¹. In any case, a current response accompanying the redox of [Fe(CN)₆]³⁻ was observed, and the electrode cell operated as an electrode cell. The results of the above CV measurement are shown in FIG. 26.

### REFERENCE SIGNS LIST

10 body fat percentage measuring device
11 composite film
12 AC power supply device
13 impedance measuring unit
20 and 50 sensor element
22, 221, 222 and 223 molecular recognition bodies
23 gap
24 substrate
30 two-electrode cell
31 base material
32 working electrode
33 counter electrode
34 cover glass
51 flat plate electrode

1-17. (canceled)

18. A composite film comprising electroconductive nanoparticles and nanofibers, wherein
the nanofibers have a plurality of gaps therebetween that are communicated with an outside;
the electroconductive nanoparticles adhere to the surface of the nanofibers and exist in the plurality of gaps;
the nanofibers are hydrophilic and biocompatible;
the composite film is electroconductive, has a total reflectance of lower than 50% of that of a pure metal foil, and is used in close contact with a body to be contacted that is hydrophilic-treated or that contains moisture;
the composite film can achieve a moisture content of 2.5 times its dry weight; and
a change in the resistance value of the composite film caused by an increase or decrease of a liquid existing in the plurality of gaps is 0.5Ω or smaller.

19. The composite film according to claim 18, wherein the amount of the electroconductive nanoparticles is 2.0 to 20 vol. % with respect to the total amount (100 vol. %) of the electroconductive nanoparticles and the nanofibers.

20. The composite film according to claim 18, wherein the nanofiber comprises cellulose.

21. The composite film according to claim 18, wherein the electroconductive nanoparticle comprises a metal, a metal oxide, or carbon.

22. The composite film according to claim 18, wherein a tensile strength of the composite film is 0.5 to 100 MPa.

23. The composite film according to claim 18, wherein the body to be contacted is skin or a tissue in a living body.

24. The composite film according to claim 18, wherein the body to be contacted comprises metal, glass, plastic, ceramic, or carbon.

25. The composite film according to claim 18, wherein
the composite film has flexibility due to which the composite film is deformed or expands or contracts in accordance with the movement of the human body when having been attached to the human body, and shows a change in the resistance value caused by the movement of the human body is 2.0Ω or smaller.

26. A sensor element comprising:
the composite film according to claim 18; and
a molecular recognition body arranged in the plurality of gaps.

27. The sensor element according to claim 26, wherein the molecular recognition body comprises an enzyme, an antibody, DNA or RNA containing an aptamer, an artificial antibody formed from a molecularly imprinted polymer, or an ion-selective molecule.

28. The sensor element according to claim 27, wherein the enzyme comprises an oxidase, a reductase, or a dehydrogenase.

29. The sensor element according to claim 28, wherein the oxidase comprises glucose oxidase or lactate oxidase.

30. The sensor element according to claim 28, wherein the dehydrogenase comprises glucose dehydrogenase or lactic acid dehydrogenase.

31. A wearable measuring device comprising the sensor element according to claim 26.

32. A body fat percentage measuring device comprising the composite film according to claim 18.

33. An electrochemical cell device comprising the composite film according to claim 18.
